(19) United States
(12) Patent Application Publication
(10) Pub. No.: US 2018/0256487 A1
(43) Pub. Date: Sep. 13, 2018

(54) COMPOSITIONS AND METHODS FOR THE TREATMENT AND PREVENTION OF RADIATION DERMATITIS

(71) Applicant: June Jacobs Laboratories, LLC, New York, NY (US)

(72) Inventor: June Jacobs, New York, NY (US)

(21) Appl. No.: 15/760,423

(22) PCT Filed: Sep. 16, 2016
(86) PCT No.: PCT/US2016/052230

§ 371 Date: Mar. 15, 2018

Related U.S. Application Data
(60) Provisional application No. 62/220,169, filed on Sep. 17, 2015.

(51) Int. Cl. A61K 8/9789; A61Q 17/04; A61Q 19/00

(52) U.S. Cl. CPC A61K 8/9789; A61Q 19/004; A61Q 17/04

(57) ABSTRACT

The disclosure provides compositions and methods for the prophylaxis and/or treatment of radiation dermatitis or erythema.

### CROSS-REFERENCE TO RELATED APPLICATION

The current application claims priority to U.S. Provisional Patent Application Ser. No. 62/220,169 filed on Sep. 17, 2015 and entitled “Cream for the Treatment and Prevention of Radiation Dermatitis,” which is incorporated by reference herein in its entirety.

### FIELD OF THE INVENTION

The present invention relates to compositions and methods for the treatment and prevention of radiation dermatitis and associated skin conditions.

### BACKGROUND OF THE INVENTION

Radiation treatment may cause a variety of adverse skin reactions which result in pain, discomfort, irritation, itching, and burning. Radiation induced skin changes can affect activities of daily living and quality of life. Individuals may experience difficulties with wearing or managing their usual clothing, restriction in the movement of a limb or affected area, visible reactions from others, loss of independence and self-care, and incur costs in managing some skin reactions. Adverse skin reactions are experienced by up to 95% of patients. Goals of care related to the management of radiation exposed skin reactions include maintaining skin integrity, cleanliness, comfort, and the reduction of pain, protection from trauma, prevention and management of infection, and the promotion of a moist wound healing environment.

Prior treatment includes washing of the affected area and application of various non-specialized creams and lotions, and/or barrier films. Such topical treatments include ingredients such as aloe vera, trolamine, hyaluronic acid, calendula, corticosteroids, and sulcrafate. Clinical studies assessing these various standard treatments for use in treatment of radition dermatitis indicate these treatments have been minimally effective.

There is a need for more effective prophylaxis and treatment of radiation dermatitis.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### SUMMARY OF THE INVENTION

The present invention provides compositions and methods for the treatment and prevention of radiation dermatitis and associated erythema. The compositions and methods described herein may also be utilized for daily application to asymptomatic skin to increase skin moisturization and moisture retention, enhance skin softness, and reduce wrinkles and other signs of aging.

The disclosure provides compositions comprising at least two skin soothing agents, a blend of at least two antioxidants, at least one skin conditioning agent, and a cosmetically acceptable vehicle. Soothing agents may make up about 2% to 15% or 5% to 10% of the composition by weight of the composition. Skin soothing agents may be selected from the group consisting of aloe vera, phytostearyl canola glycerides, allantoin, caprylic/capric triglyceride, bisabolol, colloidal oatmeal, Peucedanum ostruthium leaf extract, a long chain alkyl benzoate such as C₁₂₋₁₅ alkyl benzoate, C₁₆₋₁₇ alkyl benzoate, stearyl benzoate, isostearyl benzoate, ethylhexyl benzoate, octyldodecyl benzoate, and a ceramide. Skin soothing agents may comprise about 1.5% of the composition by weight. Compositions of the present invention may comprise at least four skin soothing agents. The at least four skin soothing agents comprise about 1.5% to 2% of the composition by weight. Skin soothing agents comprise at least two or at least four of the following: phytostearyl canola glycerides, bisabolol, Peucedanum ostruthium leaf extract, and ceramide AP.

The compositions described by the present disclosure may comprise a blend of at least four antioxidants selected from Leontopodium alpinum extract, Aspalathus linearis (red tea) leaf extract, Camellia sinensis (green and white Tea) leaf extract, Lycium varbarum (Goji Berry) fruit extract, Punica granatum (Pomegranate) extract, and Vitis vinifera (Grape) seed extract. Antioxidant blends may include Leontopodium alpinum extract. Antioxidant blends may comprise about 0.8% to 1.2% of the composition by weight.

In some embodiments, the at least one skin conditioning agent is selected from Butyrospermum parkii (Shea) butter, triolein, and argania spinosa kernel oil. In some embodiments, the at least one skin conditioning agent is selected from triolein, and argania spinosa kernel oil. In some embodiments, the at least one skin conditioning agent comprises from about 2% to 10% or from about 2% to 8% or from about 8% to 10% of the composition by weight.

The compositions described by the present disclosure may comprise at least one emulsifier. At least one emulsifier may be selected from oleic acid, palmitic acid, glyceryl stearate, cetearyl alcohol, xanthan gum, linoleic acid, and lecithin.

The compositions described by the present disclosure may further comprise one or more of a skin moisturizing agent, a fragrance, an anti-microbial agent, a sunscreen, a pH modulator, and a preservative.

In some embodiments of the compositions described here, a cosmetically acceptable vehicle is an aqueous vehicle. The aqueous vehicle may be water.

The disclosure further provides methods for treating or preventing erythema and/or radiation dermatitis in a subject in need thereof. The methods may comprise topically applying a composition described herein to an affected area of the subject's skin in an amount sufficient to cover the affected area with the composition. A subject in need thereof may be a human subject. A subject in need thereof may have received radiation therapy. The subject in need thereof may be a breast cancer patient.

In some embodiments, the method further comprises reapplying the composition to the affected area once or twice daily for a period of time ranging from one to six weeks or from two to twelve weeks. An affected area may be an area that was exposed to radiation.

### BRIEF DESCRIPTION OF THE DRAWINGS

FIG. 1A-FIG. 1C show an exemplary subject questionnaire. FIG. 1A is a first page of an exemplary subject questionnaire. FIG. 1B is a second page of an exemplary subject questionnaire. FIG. 1C is a third page of an exemplary subject questionnaire.

FIG. 2 is an illustration of lighting angles used in Silflo™ Skin Replica assays.

FIG. 3 is an illustrative demonstration of Rz and Ra metrics in Silflo™ Skin Replica assays

FIG. 4A-FIG. 4D are graphs depicting results of Silflo™ Skin Replica assays shown as the variables Rz, IDL, Breadth, and NumWR vs. Visit. FIG. 4A is a graph of Rz vs. visit. FIG. 4B is a graph of IDL vs. visit. FIG. 4C is a graph of Breadth vs. visit. FIG. 4D is a graph of NumWr vs. visit.

### DETAILED DESCRIPTION

The present invention provides compositions and methods for the prophylaxis and/or treatment of radiation dermatitis or erythema. In certain embodiments, the compositions of the invention are effective to produce one or more of the following beneficial results in skin that has been exposed to radiation during a course of medical treatment: increase in skin softness, enhanced skin moisturization, improved skin tone and texture, minimized dark spots or age sports, or increased skin moisture retention.

### Compositions

The compositions of the invention comprise at least two skin soothing agents, a blend of at least four antioxidants, and at least one skin conditioning agent. The compositions may also optionally comprise one or more of a skin moisturizing agent, an antioxidant, an emulsifier, a fragrance, an anti-microbial agent, a sunscreen, a pH modulator, and a preservative.

The compositions of the invention advantageously contain multiple skin soothing agents and antioxidants which in combination with the other ingredients (e.g., skin conditioning agent, skin moisturizing agent, and optional ingredients) are effective to soothe damaged skin and/or protect skin from radiation-induced damage, thereby treating and/or preventing at least some of the adverse skin reactions associated with radiation treatment.

In one embodiment, a composition of the invention comprises at least two, preferably at least 4 or at least 6 skin soothing agents. In one embodiment, the at least two skin soothing agents are selected from aloe vera, phytostearyl canola glycerides, allantoin, caprylic/capric triglyceride, bisabolol, colloidal oatmeal, Peucedanum ostruthium leaf extract, a long chain alkyl benzoate such as C₁₂₋₁₅ alkyl benzoate (a mixture of benzoic acid esters that consist of benzoic acid and alcohols that have carbon chain lengths from 12 to 15), C₁₆₋₁₇ alkyl benzoate, stearyl benzoate, isostearyl benzoate, ethylhexyl benzoate, octyldodecyl benzoate, and a ceramide (e.g., ceramide AP, ceramide EOP, ceramide NS, ceramide NP, ceramide NG, phytosphingosine, which can produce numerous ceramides in skin, and sphingosine). In one embodiment, the composition comprises at least 4 skin soothing agents and the at least four skin soothing agents comprise phytostearyl canola glycerides, bisabolol, Peucedanum ostruthium leaf extract, and ceramide AP.

The at least two or at least four or at least six skin soothing agents are present in an amount of from about 1.5% to 15%, about 3% to 15%, about 5% to 15%, about 7% to 15%, about 7% to 10%, or about 8% or about 10% based on total weight of the composition. In one embodiment, the at least two skin soothing agents comprises two or more of allantoin, caprylic/capric triglyceride, C₁₂₋₁₅ alkyl benzoate, bisabolol, and colloidal oatmeal in an amount of from about 1.5% to 15%, or from about 7% to 15%, or from about 10% to 15%, based on total weight of the composition. In another embodiment, the at least two skin soothing agents comprises two or more of phytostearyl canola glycerides, bisabolol, Peucedanum ostruthium leaf extract, and ceramide AP. In one embodiment the at least two skin soothing agents comprises one or more of a ceramide, aloe vera, phytostearyl canola glycerides, and Peucedanum ostruthium leaf extract in an amount of from about 0.1 to 1.5%. In one embodiment, the composition comprises at least 4 skin soothing agents and the at least four skin soothing agents comprise phytostearyl canola glycerides, bisabolol, Peucedanum ostruthium leaf extract, and ceramide AP and the sum of the at least 4 skin soothing agents is an amount of from about 1% to 2% or 1% to 1.5% based upon the total weight of the composition.

The compositions of the invention also comprise one or more skin conditioning agents or emollients. Preferably, the composition comprises from 2 to 8 or from 2 to 6 skin emollients. Emollients condition the skin by making the external layers skin (epidermis) softer and more pliable and by increasing the skin's hydration (water content) through reducing evaporation. In one embodiment the one or more emollients is selected from one or more of a glyceryl triester (e.g., trilaurin, triarachidin, tribehenin, tricaprin, tricaprylin, trierucin, triheptanoin, triheptylundecanoin, triisononanoin, triisopalmitin, triisostearin, trilinolein, trilinolenin, trimyristin, trioctanoin, triolein, tripalmitin, tripalmitolein, triricinolein, tristearin, triundecanoin, glyceryl triacetyl hydroxystearate, glyceryl triacetyl ricinoleate and glyceryl stearate diacetate), phytostearyl canola glycerides, squalene, neopentyl glycol dicaprylate/dicaprate-ester, dimethicone, Butyrospermum parkii (Shea) butter, argania spinosa kernel oil, glyceryl stearate, caprylyl glycol, and tridecyl trimellitate. The one or more skin conditioning agents may be present in an amount of from about 1% to 15%, about 2% to 12%, about 3% to 10%, about 4% to 10%, about 5% to 10%, or about 8% or about 10% based on total weight of the composition. In a preferred embodiment, the composition comprises from 2 to 8 emollients in an amount of from about 8% to 10%. In one embodiment, the 2 to 8 emollients comprises Butyrospermum parkii (Shea) butter, triolein, and argania spinosa kernel oil.

The compositions of the invention also comprise a blend of at least two, and preferable from four to six antioxidants. In one embodiment, the at least two antioxidants are selected from a tocopherol (e.g., tocopheryl acetate, tocopheryl linoleate, tocopheryl linoleate/oleate, tocopheryl nicotinate, tocopheryl succinate, potassium ascorbyl tocopheryl phosphate, dioleyl tocopheryl methylsilanol, and tocophersolan), Leontopodium alpinum extract, Aspalathus linearis (red tea) leaf extract, Camellia sinensis (green and white Tea) leaf extract, Lycium varbarum (Goji Berry) fruit extract, Punica granatum (Pomegranate) extract, and Vitis vinifera (Grape) seed extract. The at least two antioxidants may be present in an amount of from about 0.2% to 2.0%, about 0.2% to 1.5%, about 0.2% to 1%, or about 0.8% to 1.2%, or about 0.5%, about 0.8%, or about 1% based on total weight of the composition. In a preferred embodiment, a composition of the invention comprises at least two different antioxidants, preferably from 4 to 6 different antioxidants, in an amount of from about 0.8% to 1.2% based on total weight of the composition. In one embodiment, the at least two different antioxidants comprises Leontopodium alpinum extract. In one embodiment, the composition comprises 4 to 6 different antioxidants, at least one of which is Leontopodium alpinum extract and the remainder are selected from Aspalathus linearis (red tea) leaf extract, Camellia sinensis (green and white Tea) leaf extract, Lycium varbarum (Goji Berry) fruit extract, Punica granatum (Pomegranate) extract, and Vitis vinifera (Grape) seed extract.

The compositions of the invention may also comprise one or more additional optional ingredients. The optional ingredients may be selected from one or more of a skin moisturizing agent, an emulsifier, a fragrance, an anti-microbial agent, a sunscreen, a pH modulator, and a preservative as described in more detail in the following paragraphs.

The compositions of the invention also optionally comprise one or more skin moisturizing agents. In one embodiment the one or more skin moisturizing agents is selected from the group consisting of dipotassium glycyrrhizinate, pantothenic acid, and sorbital.

The compositions of the invention may also optionally comprise one or more emulsifiers. In one embodiment, the one or more emulsifiers is selected from a fatty acid, e.g., stearic acid, oleic acid, lauric acid, palmitic acid and myristic acid. In one embodiment, the one or more emulsifiers is selected from oleic acid, palmitic acid, glyceryl stearate, cetearyl alcohol, xanthan gum, linoleic acid, and lecithin. The one or more emulsifiers may be present in an amount of from about 0.1% to 1%, of from about 0.1% to 0.5%. In one embodiment, the composition comprises at least two emulsifiers. In one embodiment, the at least two emulsifiers comprise or consist of oleic acid, linoleic acid and palmitic acid.

The compositions of the invention may also optionally comprise one or more preservatives. Preferably, the preservative is effective at neutral pH levels and is a non-paraben preservative. In one embodiment, the preservative is selected from one or more of calcium disodium EDTA, diammonium EDTA, dipotassium EDTA, disodium EDTA, TEA-EDTA, tetrasodium EDTA, tripotassium EDTA, trisodium EDTA, HEDTA (hydroxyethyl ethylenediamine triacetic acid) and its trisodium salt, trisodium HEDTA, potassium citrate, aluminum citrate, diammonium citrate, ferric citrate, magnesium citrate, monosodium citrate and zinc citrate, tributyl citrate, triethyl citrate, tri-C₁₂₋₁₃ alkyl citrate, tri-C₁₄₋₁₅ alkyl citrate, tricaprylyl citrate, triethylhexyl citrate, triisocetyl citrate, trioctyldodecyl citrate and yriisostearyl citrate. In one embodiment, the preservative is selected from one or more of sodium benzoate, potassium sorbate, citric acid, disodium EDTA and caprylhydroxamic acid.

The compositions of the invention may also optionally comprise one or more pH modulators. The one or more pH modulators may be selected from among those known in the art, for example sodium hydroxide, calcium hydroxide, magnesium hydroxide and potassium hydroxide.

The compositions of the invention may also optionally comprise one or more anti-microbial agents. In one embodiment the one or more anti-microbial agents is selected from radish root ferment filtrate (leuconostoc) and algae extract, or both.

The compositions of the invention may also optionally comprise one or more sunscreens. The optional sunscreen for use in the compositions of the invention is preferably a natural sunscreen such as aloe vera or an extract thereof. In one embodiment the aloe vera extract comprises one or more of anthraquinone glycosides, polysaccharides, sterols, gelonins, and chromones. Other sunscreens may also optionally be used, including without limitation derivatives of PABA, cinnamate and derivatives of salicylate (other than ferulyl salicylate), e.g., octyl methoxycinnamate and 2-hydroxy-4-methoxy benzophenone (also known as oxybenzone).

In connection with the ingredients described herein as plant extracts, one or more of the flower, leaves, stems, and roots of the plant may be processed into the extract. In one embodiment, the extract is a polyphenol extract. In one embodiment, the extract is from the leaves of the plant.

In addition to the optional ingredients discussed above, the compositions of the invention may also comprise one or more additional optional ingredients including a thickener, coloring agent, or powder.

A thickener may be present in amounts anywhere from about 0.1% or less to about 20% or more by weight, such as from about 0.5% to about 10% by weight of the composition. Exemplary thickeners may be cross-linked polyacrylate materials available under the trademark Carbopol. Gums may be employed such as xanthan, carrageenan, gelatin, karaya, pectin and locust beans gum. Under certain circumstances the thickening function may be accomplished by a material also serving as a silicone or emollient. For instance, silicone gums in excess of 10 centistokes and esters such as glycerol stearate have dual functionality.

Powders may be incorporated into the cosmetic composition of the disclosure. These powders include chalk, talc, kaolin, starch, smectite clays, chemically modified magnesium aluminum silicate, organically modified montmorillonite clay, hydrated aluminum silicate, fumed silica, aluminum starch octenyl succinate and mixtures thereof.

### Cosmetically Acceptable Vehicles

The compositions described here may optionally include one or more cosmetically acceptable vehicles. The vehicle may act as a dilutant, dispersant or carrier for the active ingredients, so as to facilitate their distribution and uptake when the composition is applied to the skin. The most common vehicle is water and water is the preferred vehicle for the present compositions. Vehicles other than or in addition to water can include liquid or solid emollients, solvents, humectants, and thickeners, e.g., glycerin, butylene glycol, propylene glycol, water, various oils (jojoba, sweet almond, soybean, sunflower, apricot, etc.), and the like. The vehicle may comprise from about 25% to 75% of the composition by weight. In one embodiment, the vehicle makes up about 50% of the composition by weight.

The compositions described herein may be formulated as aqueous, aqueous/alcoholic or oily solutions; dispersions of the lotion or serum type; anhydrous or lipophilic gels; emulsions of liquid or semi-liquid consistency, which are obtained by dispersion of a fatty phase in an aqueous phase (O/W) or conversely (W/O); or suspensions or emulsions of smooth, semi-solid or solid consistency of the cream or gel type. These compositions are formulated according to the usual techniques as are well known to this art.

When the compositions of the invention are formulated as an emulsion, the proportion of the fatty phase may range from about 5% or less to about 80% or more by weight, such as from about 10% to about 50% by weight, relative to the total weight of the composition. Oils, emulsifiers and co-emulsifiers incorporated in the composition in emulsion form are selected from among those used conventionally in the cosmetic or dermatological field. The emulsifer and coemulsifier may be present in the composition at a proportion ranging from about 0.3% or less to about 30% or more by weight, such as from about 0.5% to about 20% by weight, relative to the total weight of the composition.

When the compositions of the invention are formulated as an oily solution or gel, the fatty phase may constitute more than about 50% or less, more than about 60%, more than about 70%, more than about 80%, more than about 90% of the total weight of the composition.

The cosmetic compositions described here may be formulated in any form suitable for application to the site of interest, including a lotion, cream, gel, shampoo, etc. The composition can be packaged in a suitable container to suit its viscosity and intended use by the consumer. For example, a lotion or cream can be packaged in a bottle, or a propellant-driven aerosol device or a container fitted with a pump suitable for finger operation. When the composition is a cream, it can simply be stored in a non-deformable bottle or squeeze container, such as a tube or a lidded jar. The disclosure accordingly also provides a closed container containing a cosmetically acceptable composition as herein defined.

### Methods

The present invention provides methods for treating erythema and/or radiation dermatitis. The invention also provides methods for the treatment or prevention of skin conditions associated with erythema and/or radiation dermatitis including but not limited to dryness, redness, itching and inflammation. In accordance with the methods described here, the compositions of the invention are effective to produce one or more of the following beneficial results in skin that has been exposed to radiation during a course of medical treatment: increase in skin softness, enhanced skin moisturization, improved skin tone and texture, minimized dark spots or age sports, and increased skin moisture retention.

In certain embodiments of the methods described here, a composition of the invention is applied to the skin in a daily regimen that may include pre-application for one or more days before radiation exposure. In accordance with one aspect of the methods described here, a composition of the invention is applied to the skin for a period of time following radiation exposure. In accordance with another aspect, a composition of the invention is applied to skin in need of treatment for dryness and/or hardness, for example as may be caused by radiation dermatitits, including chronic radiation dermatitis, or erythema.

In one embodiment, the invention provides methods for improving one or more of skin moisturization, skin tone, skin texture, and skin moisture retention, the method comprising applying a composition as described herein to the skin of a subject in need thereof at least once daily. In one embodiment, the subject in need thereof is a subject undergoing radiation therapy, e.g., for cancer treatment. Compositions of the present invention may be administered prior to beginning a course of radiation therapy and throughout the course of treatment to aid in the prevention of developing radiation dermatitis. Furthermore, the compositions of the invention may be administered to a subject that has undergone radiation therapy and may have already developed radiation dermatitis to aid in the treatment of the dermatitis.

Compositions of the invention may administered to an affected area once daily, twice daily, three times daily, once every two days, once every three days, or once a week. Administration may be continued for about one week, about two weeks, about three weeks, about four weeks, about five weeks, about six weeks, about seven weeks, about eight weeks, about weight weeks, about 12 weeks, about 6 months, about a year, or indefinitely.

Methods of measuring the effectiveness of compositions and methods of the present invention may rely on one or several objective measures of skin integrity. Evaluation of skin texture may involve image analysis of skin replicas in a silicon resin (SILFLO®). As with a dental impression, skin replicas provide details of a skin surface in a hardened model. Image analysis of a skin replica include, for example, assessment of the shadows cast within the skin as a measure of depth and magnitude of skin creasing or wrinkling.

Another test utilized to assess skin integrity is the use of evaporative water loss measurements obtained under steady-state. These measurements are taken following an acclimation period in a controlled temperature and humidity environment. These measurements can be made using a DermaLab® System (Cortex Technology) equipped with dual calibrated TEWL probes. This technology is based on the vapor pressure gradient estimation method. The probes contain two sensors that measure the temperature and relative humidity at two fixed points along the axis normal to the skin surface. This arrangement is such that the device can electronically derive a value that corresponds to evaporative water loss expressed in grams per square meter per hour (g/m² h).

Skin surface hydration may be measured using a Corneometer®. The Corneometer® is a commercially available instrument (Courage and Khazaka, Germany) that is designed to measure changes in the capacitance of the skin resulting from changes in the degree of hydration. It is particularly sensitive to low hydration levels. The Corneometer® expresses the capacitance of the skin in arbitrary units of skin hydration (H). Measurements are obtained by holding the probe against the surface of the skin.

Skin surface hydration, transepidermal water loss, or skin creasing/wrinkling and other measures of skin integrity and quality may be improved by the compositions and methods of the present invention as objectively measured by known methods, including those described above.

### Radiation Therapy

Radiation therapy is a long-established and effective component of modern cancer therapy for localized disease. However, the ultimate utility of radiation therapy is limited by the fact that some cancer cells are resistant to ionizing radiation. Additionally, the delivery of the ionizing radiation through healthy tissue or beyond the tumor margin limits the radiation dose and may result in unwanted side effects.

Radiation therapy may be utilized to treat a variety of cancers including breast cancer. Radiation therapy may be appropriate at any stage of breast cancer treatment. For example, radiation therapy may be utilized in the treatment of subjects with stage 0 through stage IV breast cancer. Radiation therapy may be utilized following lumpectomy or mastectomy.

Radiation therapy is not recommended when radiation to a particular body region has reached a suggested dose, in women who are pregnant, at sites of extreme sensitivity, or in incidents where a subject cannot adhere to a radiation schedule.

Radiation therapy may include external radiation, internal radiation, or intraoperative radiation. The unit used to measure radiation therapy dosage is the Gray (Gy). Typical radiation therapy dosage for treating breast cancer is 45 to 60 Gy. Radiation therapy is typically dose fractioned such that radiation is administered at a number of treatments over a period of one to two months. In some cases, radiation therapy is administered daily, 5 days a week for a total period of five to eight weeks.

### Erythema

Erythema is a condition resulting in redness or rash of the skin or mucous membranes. Erythema may also be described as hyperemia due to the involvement of superficial capillaries. There are many causes of erythema, including by way of example, infection, medications (e.g. birth control, sulfa drugs, penicillin, anti-seizure medications, nonsteroidal anti-inflammatories), lupus, pregnancy, ulcerative colitis, Bechet's disease, Crohn's disease, etc. Erythema may be coincident with radiation dermatitis.

### Radiation Dermatitis

Acute Radiation Dermatitis occurs within 90 days of exposure. The patient may have changes ranging from faint erythema and dry desquamation to skin necrosis and ulceration, depending on the severity of the reaction. The National Cancer Institute has developed a 4 stage criteria for the classification of acute radiation dermatitis:

Grade 1—Faint erythema or dry desquamation.

Grade 2—Moderate to brisk erythema or patchy, moist desquamation confined to skin folds and creases. Moderate edema.

Grade 3—Confluent, moist desquamation greater than 1.5 cm diameter, which is not confined to the skin folds. Pitting edema.

Grade 4—Skin necrosis or ulceration of full thickness dermis.

Chronic Radiation Dermatitis is an extension of the acute process and involves further inflammatory cytokines. Long-lasting impairment of the skin's ability to heal can be due to compromised cellular dysfunction. Fibroblasts may be permanently altered, leading to atrophy and fibrosis.

The invention can be further understood by reference to the following examples, which are provided by way of illustration and are not meant to be limiting.

### EXAMPLES

### Example 1

### Clinical Study

### Study Design

A clinical study was performed to test the efficacy of a composition falling within the scope of the present invention (herein referred to as “healing cream”). The objectives of the study were:
1. to assess the effect of a healing cream on skin surface hydration (moisturization) and skin barrier function;
2. to determine the ability of a healing cream to improve facial skin texture;
3. and to obtain the consumer opinion of a healing cream during an eight week use period in healthy female subjects.

Study Schedule

A total of 55 female subjects were selected to participate in the evaluation of a healing cream, which consisted of Corneometer® measurements for the evaluation of skin surface hydration {moisturization}, DermaLab® measurements of transepidermal water loss (TEWL) {skin barrier function}, Silflo™ skin replicas with image analysis for the evaluation of skin texture, and consumer perception questionnaires at designated study intervals. A study schedule appears below in Table 1.

TABLE 1
Study Schedule

| | | | 24 | Week | Week | Week |
|---|---|---|---|---|---|---|
| Procedure/Evaluation | Screening | Baseline | Hours | 2 | 4 | 8 |
| Informed Consent, | X | | | | | |
| Inclusion/Exclusion | | | | | | |
| Medical history/ | X | | | | | |
| concomitant | | | | | | |
| medications | | | | | | |
| Purpose ® bar | X | | | | | |
| soap distribution | | | | | | |
| Corneometer ® | | X | X | | | |
| measurements | | | | | | |
| DermaLab ® TEWL | | X | X | X | X | X |
| measurements | | | | | | |
| Silflo ™ skin | | X | | X | X | X |
| replica sampling | | | | | | |
| Test material, | | | X | | | |
| application instructions | | | | | | |
| and daily diary | | | | | | |
| distribution | | | | | | |
| Test material | | X | | | | |
| application to the | | | | | | |
| desianated forearm | | | | | | |
| at the laboratory | | | | | | |
| Health history/ | | X | X | X | X | X |
| medication update | | | | | | |
| Consumer perception | | | | X | X | X |
| questionaire | | | | | | |
| Test material and daily | | | | | | X |
| diary collection | | | | | | |

Testing Material

The testing material (healing cream) was provided by June Jacobs Spa Collection and given an anonymous identification number by Clinical Research Laboratories, Inc.

Test materials were stored at room temperature and humidity prior to distribution to subjects. All remaining test materials will be retained by Clinical Research Laboratories, Inc. for a period of 6 months.

Application Instructions

Study participants were provided with the following application instructions:

Face: Apply twice daily, morning and night.

Forearm: Apply only to the designated forearm twice daily, morning and night.

Study Population

A total of 55 female subjects, ranging in age from 32 to 65 years, were selected for the study (Subject Demographics-Appendix I). A total of 13 Subjects (˜24%) reported sensitive skin and 42 subjects (˜76%) reported normal skin. Subjects who met all of the inclusion criteria and none of the exclusion criteria listed in the study protocol were enrolled for participation.

Inclusion Criteria

A subject may be eligible to participate in this study if all of the following conditions are met:

a. Subject is female between 18 and 65 years of age;

b. Subject reports self-perceived sensitive skin (approximately 25% of the study population) or normal skin (approximately 75% of the study population);

c. Subject agrees to refrain from application of any products to the test sites, with the exception of the provided test material and soap, for the duration of the study (including skin treatment products, cleansers, moisturizers, serums, toners, masks, soaps, and sunscreens);

d. Subject is free from any skin (dermatological or systemic) disorders, which, in the opinion of the Investigator, would interfere with the test results or increase the risk of adverse reaction;

e. Subject is willing to avoid extended periods of sun exposure for the duration of the study (including artificial tanning);

f. Subject agrees not to introduce any new cosmetic or toiletry products during the study;

g. Subject is dependable and able to follow directions as outlined in the protocol;

h. Subject is in generally good health, and has a current Panelist Profile/Medical History Form on file;

i. Subject has completed a HIPAA Authorization Form in conformance with 45 CFRParts 160 and 164;

j. Subject understands and is willing to sign an Informed Consent in conformance with 21 CFR Part 50: “Protection of Human Subjects.”

Exclusion Criteria

A subject may be considered ineligible to participate in this clinical study if any of the following conditions are met:

a. Subject is pregnant, nursing or planning a pregnancy;

b. Subject is currently using medications or oral supplements, such as systemic or topical corticosteroids, anti-inflammatory drugs, antihistamines or retinoid medications or products which, in the opinion of the Investigator, may influence the outcome of the study or interfere with study observations;

c. Subject has known allergies to cosmetics or toiletry products;

d. Subject exhibits sunburn, rashes, scratches, burn marks, etc. on the test sites, which might interfere with study evaluations;

e. Subject reports a history of acute or chronic dermatological, medical, and/or physical conditions that would preclude application of the test material(s) and/or could influence the outcome of the study;

f. Subject has participated in a clinical study which involved the test sites within one week of study initiation.

Informed Consent

The study procedures will be explained to all subjects intending to participate. All subjects will be completely informed about the pertinent details and purpose of the study, according to the informed consent guidelines. Subjects will be given the opportunity to ask questions. Two copies of an Informed Consent Form will be read and signed by each subject. One copy will be given to the subject and the other will be retained in the study file.

Subject Withdrawal

After admission to the study, the subject may withdraw at any time for any reason, but the reason must be reported fairly and accurately.

Randomization

Designation of the right or left side of the face for Silflo™ skin replicas will be determined by a computer-generated randomization code. Designation of the right or left volar forearm for test material application will be determined by a separate computergenerated randomization code. The contralateral forearm will remain untreated. The assigned application/measurement sites will remain consistent throughout the study.

Blinding

Unless otherwise designated by the Sponsor, subjects will not be provided with information regarding the identity of the test material(s). The investigatory staff will not be blinded. Test materials will be labeled with a unique CRL study identification number, a panel code and subject numbers in accordance with distribution.

Study Evaluations/Instrumentation—Corneometer® Measurements {Skin Surface Hydration}

Skin surface hydration will be measured using the Corneometer®. The Corneometer® is a commercially available instrument (Courage and Khazaka, Germany) that is designed to measure changes in the capacitance of the skin resulting from changes in the degree of hydration. It is particularly sensitive to low hydration levels. The Corneometer® expresses the capacitance of the skin in arbitrary units of skin hydration (H). Measurements are obtained by holding the probe against the surface of the skin. Readings are displayed on an LCD then manually transcribed onto a score sheet. Three consecutive readings will be obtained from the test site and averaged. Increases in posttreatment Corneometer® readings are indicative of a hydrating effect resulting from increased skin moisture content. Decreases in Corneometer® readings indicate a drying effect resulting from decreased moisture content, and consistent Corneometer® readings suggest stabilization of skin moisture content, which can be described as a “non-drying” effect.

Corneometer® measurements will be obtained from the center of each test site on the volar aspect of the forearms.

Study Evaluations/Instrumentation—DermaLab® Measurements (TEWL) {Skin Barrier Function}

Evaporative water loss measurements obtained under steady-state conditions provide an instrumental assessment of skin barrier function. These measurements are taken following an acclimation period in a controlled temperature and humidity environment. These measurements can be made using a DermaLab® System (Cortex Technology) equipped with dual calibrated TEWL probes. This technology is based on the vapor pressure gradient estimation method. The probes contain two sensors that measure the temperature and relative humidity at two fixed points along the axis normal to the skin surface. This arrangement is such that the device can electronically derive a value that corresponds to evaporative water loss expressed in grams per square meter per hour (g/m² h).

Measurements are obtained by holding the probe against the surface of the skin. Three 60-second readings will be taken from the same area within each test site and averaged. Readings are electronically displayed and then manually transcribed onto a score sheet. Excessively high TEWL values indicate a disruption of the skin barrier, stabilized TEWL values are indicative of an intact skin barrier, and decreased TEWL values suggest improved skin barrier function.

DermaLab® measurements will be obtained from the center of each test site on the volar aspect of the forearms.

Study Evaluations/Instrumentation—Image Analysis of Silflo™ Skin Replicas {Skin Texture}

The texture of the skin, specifically qualities of facial lines in the lateral canthal area, can be assessed by making negative impressions of the skin. For the crow's feet area, skin replicas are made by placing Silflo® impression material against the sampling area, positioned with Replica™ locating rings. Replicas are made in the same manner for all subjects by positioning the locating ring, filled with Silflo® paste, in consistent alignment for every sample taken during the study period. Locating rings are positioned such that the tab of the ring is directed toward the top of the head of each subject.

The resultant replicas can be evaluated by a technique that combines image analysis and surface shadowing under grazing illumination. The replicas are illuminated at a precisely defined angle to create shadows that are analyzed according to shades of gray. In order to analyze the same area from replicas before and after treatment, a mask of the before treatment replica is made and stored digitally. This mask is then superimposed on the after-treatment replica for positioning (Grove et al. 1989, Sun et al. 1997, Hong-Keun and Young-Hwan 1997).

Study Evaluations/Instrumentation—Questionnaires

Following product use, an assessment of the effects of a test material can be determined by questioning the treated subjects. Questionnaire administration using the eListen 5.5 Electronic Survey and Data Collection Tool allows for an efficient and accurate method of determining subject response proportions to assess the consensus opinion of a clinical study population.

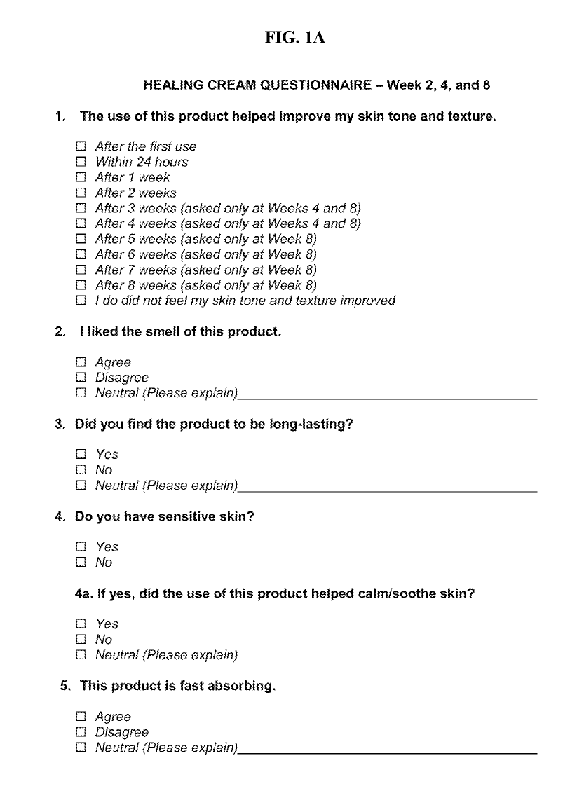

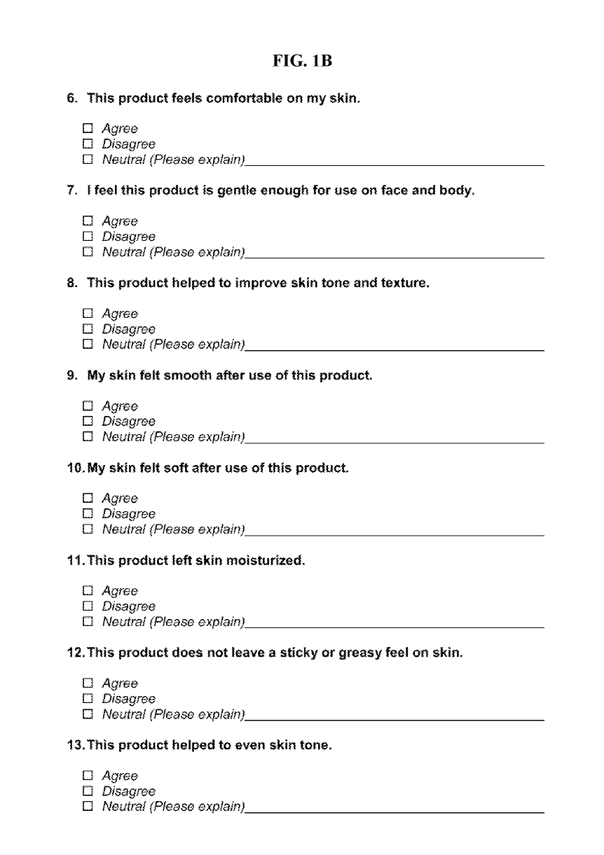

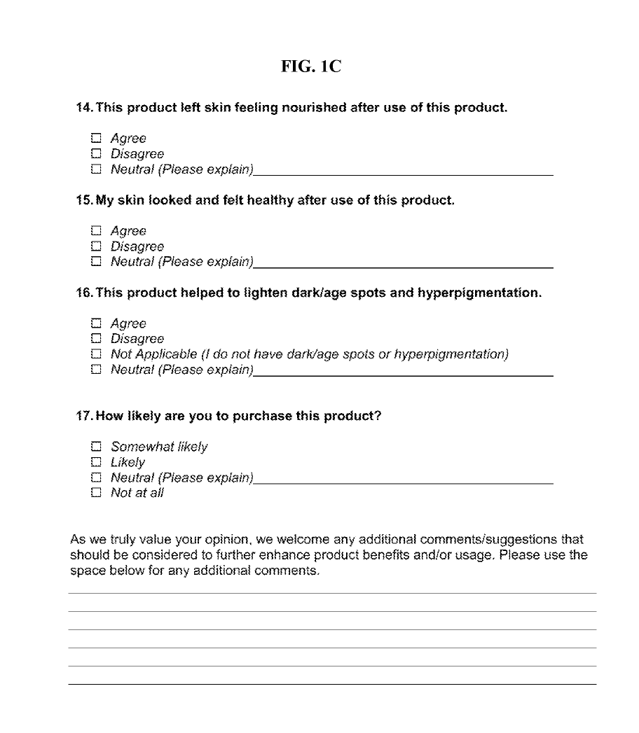

A sample Questionnaire is provided in FIG. 1A-FIG. 1C.

Subject Identification

Candidates for study participation will be identified from the Clinical Research Laboratories, Inc. (CRL) database. All subjects will be initially identified by a permanent CRL Identification Number. Subjects who meet the qualification criteria will be assigned a study subject number. This subject number will be assigned in sequence as subjects are enrolled in the study. A master roster will be kept of the permanent CRL Identification Number and the corresponding study subject number.

Screening Visit

Subjects will arrive at the CRL testing facility for the screening visit with a clean face, free of make-up. Inclusion/Exclusion Criteria will be verified and informed consent obtained. A designated study staff member will collect concomitant medication and medical history information from each subject. Subjects who meet all the study requirements will be enrolled.

To standardize the skin condition of the study population at baseline and minimize variability attributable to use of different skin care regimens, subjects will participate in a 7-day (±3 days) conditioning period.

Each subject will be provided with written study instructions and a bar of Purpose® soap to use at least once daily on the face and forearms and for all facial and body cleansing during the conditioning phase and during the eight week study. Subjects will also be required to discontinue use of all skin treatment products on the face and forearms for the duration of the study.

Baseline Visit

Subjects will arrive at the CRL testing facility for the baseline visit with a clean face and forearms, free of any make-up or lotion. Subjects will be queried regarding health and medication changes and will acclimate to ambient laboratory conditions for a period of 15 minutes prior to study evaluations/instrumentation.

Following the acclimation period, a CRL Technician will identify and outline one 2.5″×2.5″ test site on the volar aspect of each forearm utilizing an acetate template and gentian violet marker. The following assessments will then be performed:

Skin surface hydration—Corneometer® measurements [forearms] (section 9.1)

Transepidermal water loss—DermaLab® measurements [forearms] (section 9.2)

Skin replicas-Silflo™ impressions [face] (Section 9.3)

The location of the sampling area for skin replicas will be documented by measuring the distance from anatomical landmarks and recorded on a test site locator document.

A CRL technician will apply the test material to the designated forearm, according to the randomization schedule. Subjects will not be permitted to apply any products to the forearms and will be instructed to refrain from showering or getting the arms wet until after the 24 hour visit.

24 Hour Visit

Subjects will return to the CRL testing facility 24 hours (±one hour) following the initial application to the designated forearm. Subjects will be queried regarding health and medication changes and will acclimate to ambient laboratory conditions for a period of 15 minutes prior to study evaluations. Following the acclimation period, the following assessments will be performed:

Skin surface hydration—Corneometer® measurements [forearms] (section 9.1)

Transepidermal water loss—DermaLab® measurements [forearms] (section 9.2)

Subjects will be provided with the test material, application instructions (Section 3.2), and a daily diary in which to record use of the test material and any subjective comments. Subjects will be instructed to refrain from using any other skin treatment products or soap on the face or forearms, with the exception of those provided, for the duration of the study.

Week 2 and Week 4 Visits

Subjects will return to the CRL testing facility following approximately two and four weeks of test material use, having applied the test material to the face and designated forearm 2 hours (±30 minutes) prior to the assigned appointment time. Subjects will be queried regarding health and medication changes. Daily diaries will be reviewed and subjects will complete a consumer perception questionnaire (section 9.4 & Appendix I) while acclimating to ambient laboratory conditions for a period of 15 minutes prior to study evaluations/instrumentation.

Following the acclimation period, the following assessments will be performed:

Transepidermal water loss—DermaLab® measurements [forearms] (section 9.2)

Skin replicas-Silflo™ impressions [face] (Section 9.3)

At each visit, the acetate template used at the Baseline Visit will be positioned on each forearm to identify measurement areas and the test site locator document will be referenced to ensure proper positioning of Silflo™ replica material on the designated crow's feet area.

Week 8 Visit

Subjects will return to the CRL testing facility following approximately eight weeks of test material use, having applied the test material to the face and designated forearm 2 hours (±30 minutes) prior to the assigned appointment time. Subjects will be queried regarding health and medication changes. Test materials and daily diaries will be collected, and daily diaries will be reviewed. Subjects will complete a consumer perception questionnaire (section 9.4 & Appendix I) while acclimating to ambient laboratory conditions for a period of 15 minutes prior to study evaluations/instrumentation.

Following the acclimation period, the following assessments will be performed:

Transepidermal water loss—DermaLab® measurements [forearms] (section 9.2)

Skin replicas-Silflo™ impressions [face] (Section 9.3)

The acetate template used at the Baseline Visit will be positioned on each forearm to identify measurement areas and the test site locator document will be referenced to ensure proper positioning of Silflo™ replica material on the designated crow's feet area.

Adverse Events

All adverse events will be documented in the subject's source documentation and reported to the Sponsor promptly.

An adverse event is any untoward medical occurrence, whether or not it is considered study related, including death, experienced by a subject. An event may consist of a disease, an exacerbation of a pre-existing illness or condition, an occurrence of an intermittent illness or condition, a set of related symptoms or signs, or a single symptom or sign.

An adverse event would include:

1. Any new events not present before the initial enrollment into the study.

2. A pre-existing event that recurs with increased intensity (severity) or frequency subsequent to initial enrollment into the study.

3. An event that is present at the time of enrollment that exacerbates following enrollment into the study.

A “serious” adverse event is:

1. Death due to any cause, whether or not it is felt to be related to the study.

2. Life-threatening event-an event is present when the subject was, in the view of the Investigator, at immediate risk of death from the event as it occurred. Note that this definition does not include an event that, had it occurred in a more serious form, might have caused death.

3. Any event that is disabling or incapacitating.

4. Events that require or prolong subject hospitalization. Hospitalization for elective treatment of a pre-existing condition is not considered a “serious” adverse event.

Each adverse event will be promptly recorded and sufficiently documented by the Investigator or designee in the source documentation/case report form even if the adverse event is assessed as unlikely to be related to the study. Details recorded will include the nature of the adverse event, onset date/time, duration, severity, outcome and relationship to test product. All adverse events will be followed up until resolved, stabilized, the subject is lost to follow-up or the event is otherwise explained. All follow-up information should be recorded Any adverse event requiring medical attention will be referred to appropriate CRL medical personnel.

The Investigator or designee will report the occurrence of any serious adverse event to the Sponsor in writing, within one business day regardless of the causal relationship to the study and follow-up with written documentation within three business days.

Amendments/Deviations

Neither the testing facility nor the Sponsor will modify this protocol without prior written notification. Protocol deviations should be avoided whenever possible. All deviations will be documented in the study file.

Statistical Analysis

Analysis of variance followed by Dunnett's test will be applied to determine the differences between baseline and each post-treatment interval for each evaluation parameter (Corneometer® measurements and DermaLab® measurements). Change from baseline will be calculated at each post-treatment time point.

Statistical significance exists for two-sided p-values ≤0.05 at the 95% significant level.

Questionnaire responses, for which response category comparisons are informative, will be analyzed by Z-tests. Z-tests are used to determine statistically significant differences in the proportions of subjects responding positively or negatively to each question offering a range of responses. The percentage of subjects choosing the positive responses will be added together and the percentage of subjects choosing the negative responses will be added together. The split proportions are compared by calculation of a Z-Score to determine statistically significant differences.

Upon study completion, image analysis of Silflo™ Replicas will be performed by Dr. Miller at BioNET, Incorporated. A copy of the report will be included as an attachment to the final report.

Quality Assurance

Study related documents, including source documents or raw data, and final report, where applicable, will be examined for completeness, accuracy, and proper documentation practices by Clinical Research Laboratories, Inc. Quality Assurance Personnel.

Regulatory Parameters

This study will be conducted under Good Clinical Practice Guidelines, other applicable regulatory requirements, and Clinical Research Laboratories, Inc. Standard Operating Procedures. The Sponsor may conduct an on-site inspection and audit at any time during the course of the study.

Final Report

A final report will be issued four to six weeks following the conclusion of the study. The final report will summarize the study objective(s) and test methodology. Data and analysis for study evaluations and questionnaire responses, as described previously, and subject demographic information will be listed. Study conclusions will detail the results of the statistical analyses, with respect to the efficacy and consumer opinion of the test material under the conditions of the study. The final report will be signed by the Investigator, Clinical Research Laboratories, Inc. management and quality assurance/regulatory personnel conducting audit(s) of the study procedures, documentation, and/or final report.

### Test Results

A total of 47 subjects completed the 8-week study. Seven subjects (#5, #15, #16, #24, #33, #35, and #55) were lost to follow-up and one subject (#32) was discontinued due to an adverse event (see Section 9.8). Subjects #5, #32, and #55 completed the moisturization assessments and were included in analysis.

Evaluation of Skin surface Hydration [Corneometer® Measurements]

Table 2 lists individual Corneometer® measurements and calculated changes from baseline. Results of statistical analysis appear below in Table 3.

TABLE 2

| Mean Corneometer ® | | | Change from |
|---|---|---|---|
| Measurements (n = 50) | | | Baseline* |
| | | 24 | 24 |
| Treatment | Baseline | Hours | Hours |
| June Jacobs | 31.4 | 39.0 | 7.6 |
| Healing Cream | | | (p < 0.0001) |
| Untreated | 31.2 | 31.4 | 0.2 |
| Control | | | (p = 0.0171) |

*Increases in mean values compared to baseline indicate an improvement in skin surface hydration.

TABLE 3

| | Paired t-test or Wilcoxon | | | |
|---|---|---|---|---|
| | signed rank test | | Time | |
| Parameter | Treatment 1 | Treatment 2 | Interval | p-value* |
| Corneometer ® | June Jacobs | Untreated | 24 Hours- | (p < |
| Measurements | Healing Cream | Control | Baseline | 0.0001) |

*A significantly great improvement was observed for June Jacobs Healing Cream compared to the untreated Control

DermaLab® Measurements (TEWL) {Skin Barrier Function}

Table 4 lists individual DermaLab® measurements and calculated changes from baseline. Results of statistical analysis appear below in Table 5.

TABLE 4

| Mean TEWL Measurements (n = 47) | | | | | Change from Baseline* | | | | |
|---|---|---|---|---|---|---|---|---|---|
| | | 24 | Week | Week | Week | 24 | Week | Week | Week |
| Treatment | Baseline | Hours | 2 | 4 | 8 | Hours | 2 | 4 | 8 |
| June Jacobs | 7.6 | 6.0 | 5.6 | 5.3 | 4.9 | −1.6 | −2.0 | −2.3 | −2.7 |
| Healing Cream | | | | | | (p < 0.0001) | (p < 0.0001) | (p < 0.0001) | (p < 0.0001) |
| Untreated | 7.3 | 8.0 | 8.4 | 8.9 | 9.4 | 0.7 | 1.1 | 1.5 | 2.0 |
| Control | | | | | | (p < 0.0001) | (p < 0.0001) | (p < 0.0001) | (p < 0.0001) |

*Decreases in mean values compared to baseline indicate an improvement in skin barrier function for June Jacobs Healing Cream while increases in mean values compared to baseline for the untreated control indicate a disruption of skin barrier function.

TABLE 5

| | Paired t-test or Wilcoxon | | | |
|---|---|---|---|---|
| | signed rank test | | Time | |
| Parameter | Treatment 1 | Treatment 2 | Interval | p-value* |
| TEWL | June Jacobs | Untreated | 24 Hours-Baseline | (p < 0.0001) |
| Measurements | Healing Cream | Control | Week 2-Baseline | (p < 0.0001) |
| | | | Week 4-Baseline | (p < 0.0001) |
| | | | Week 8-Baseline | (p < 0.0001) |

*Significantly greater improvements were observed for June Jacobs Healing Cream compared to the untreated control.

Evaluation of Skin Texture [Image Analysis of Silflo™ Skin Replicas]

Image analysis of Silflo Skin Replicas was performed by David L. Miller, Ph. D. at BioNET, Incorporated.

Equipment utilize for analysis of Silflo™ Skin Replicas includes:

PC: IBM compatible Pentium III 500 Mhz with 1 gb memory running under Windows XP Professional.

Video: SONY solid state B&W camera, 50 mm lens/30 mm extension, Coreco TCI Ultra frame grabber.

Software: OPTIMAS v6.5, Microsoft EXCEL 2007, StatSoft STATISTICA 7.

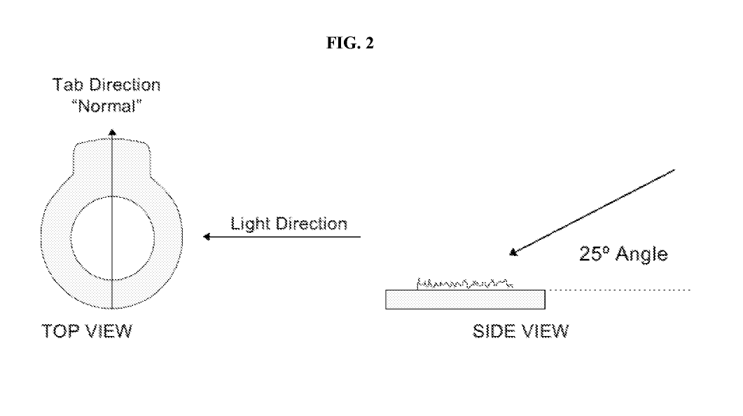

Lighting: Collimated light source directed at a 25° angle from the plane of the replica. The replica was placed in a holder that fixed the direction of the tab position of the replica so that the replica could be rotated to align the tab direction normal or parallel to the incident light direction (see FIG. 2).

The replicas were taken from the crow's feet area adjacent to each eye with the tab direction pointing toward the top of the head. The PARALLEL sampling orientation provides texture measurements sensitive to the MAJOR, expression-induced lines (crow's feet wrinkles). The NORMAL sampling orientation provides texture measurements sensitive to the MINOR, fine lines.

The general background gradient of light intensity was adjusted by applying a 1st order correction in the direction of the light propagation. The shadow texture produced by the oblique lighting of the negative replica was analyzed by two types assay methods:

A. Measuring the luminance along a set of 10 equal length parallel lines (passes) running across the replica parallel to the lighting direction. The variations in luminance were treated as indicative of the roughness and analyzed by traditional surface roughness statistics:
Rz—the average maximum difference in luminance value for five equal length segments in each of the 10 lines traversing the sample.
Ra—the average deviation of the luminance curve about the mean luminance for the same 10 lines.
The “R” parameters are reported in the units of brightness (Gray Levels) ranging from 0 to 255.
FNum—number markers per mm placed on the lines at luminance changes indicative of fine lines.
IDL—the integrated developed length of the luminance traces of the 10 scan lines.

This is the total length of the luminance lines as a proportion of the straight line distance. I.E. a flat featureless sample has an IDL of 1.000.

B. The replica image area was divided into 10 equal width bands or sub-areas. The shadow like features were detected in each of these bands according to their luminance values being less than the detection threshold. Four parameters were determined from the detected features.
Spacing—the mean distance in millimeters between adjacent detected features (i.e. spacing between the midpoints of adjacent shadowy features). If there are insufficient features in an individual replica to calculate a meaningful distance, the parameter is left blank in the data table.
Breadth—the average breadth in millimeters of the detected features in millimeters. This parameter is proportional to the depth of the wrinkle producing the shadow. If there are insufficient features in an individual replica to calculate a meaningful breadth, the parameter is left blank in the data table.
Shadows—percent of the sampled replica area with luminance values less than the detection threshold. This is the relative area of shadows cast by the wrinkles and fine lines in the replica.
NumWr—the total number of features detected in the 10 bands or sub-areas used to calculate spacing and breadth.

184 replicas were supplied for evaluation. The replicas represented 4 visit samples: BL (baseline) and W2, W4, W8 during the treatment period. A complete listing of the data is in the data section of the Appendix.

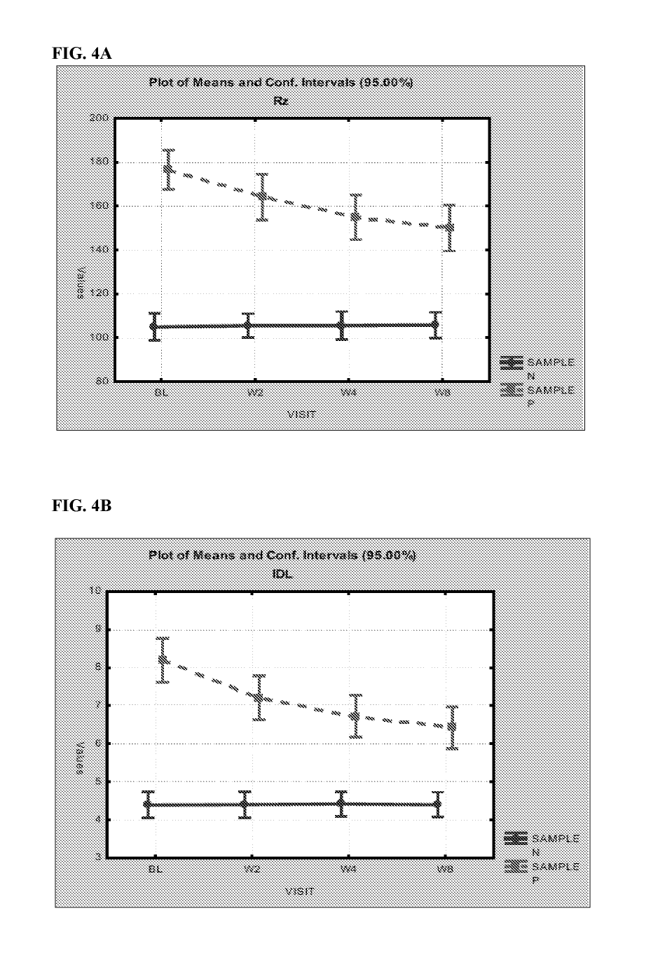

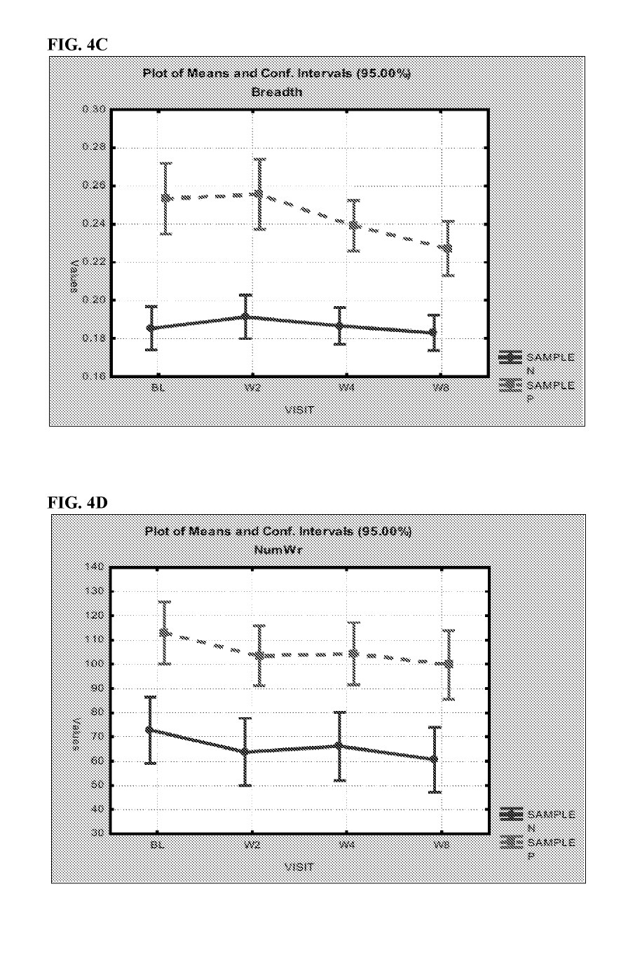

The results are summarized in Table 6 in the statistics section of the Appendix. The results portray the characteristic magnitudes for replicas made in the crow's foot are with the tab position pointing toward the top of the head: higher baseline levels of Rz, IDL, Breadth and NumWr parameters for the Parallel direction compared to the Normal direction. Graphs of the means for these variables vs Visit are shown in FIGS. 4A-4D.

TABLE 6
Breakdown Table of Descriptive Statistics

| | | | Rz | | Ra | | FNUM | | IDL | |
|---|---|---|---|---|---|---|---|---|---|---|
| SAMPLE | VISIT | N | Means | Std. Dev. | Means | Std. Dev. | Means | Std. Dev. | Means | Std. Dev. |
| N | BL | 46 | 104.9 | 20.8 | 22.4 | 4.8 | 0.544 | 0.112 | 4.393 | 1.137 |
| N | W2 | 46 | 105.5 | 18.4 | 22.4 | 3.9 | 0.525 | 0.130 | 4.403 | 1.143 |
| N | W4 | 46 | 105.6 | 21.7 | 21.5 | 4.8 | 0.541 | 0.122 | 4.417 | 1.083 |
| N | W8 | 46 | 105.8 | 19.8 | 21.6 | 4.2 | 0.515 | 0.095 | 4.400 | 1.108 |
| P | BL | 46 | 176.4 | 30.0 | 41.5 | 10.3 | 0.597 | 0.168 | 8.189 | 1.931 |
| P | W2 | 46 | 164.1 | 35.1 | 37.8 | 10.9 | 0.538 | 0.161 | 7.201 | 1.938 |
| P | W4 | 46 | 154.8 | 34.0 | 33.8 | 9.5 | 0.542 | 0.156 | 6.720 | 1.848 |
| P | W8 | 46 | 150.0 | 35.3 | 32.1 | 9.1 | 0.534 | 0.146 | 6.424 | 1.845 |
| | | | Spacing | | Breadth | | Shadows | | NumWr | |
| SAMPLE | VISIT | N | Means | Std. Dev. | Means | Std. Dev. | Means | Std. Dev. | Means | Std. Dev. |
| N | BL | 46 | 1.495 | 0.726 | 0.185 | 0.038 | 5.9 | 3.9 | 72.8 | 46.0 |
| N | W2 | 45 | 1.711 | 0.869 | 0.191 | 0.039 | 5.4 | 3.9 | 63.7 | 46.9 |
| N | W4 | 45 | 1.657 | 0.948 | 0.187 | 0.032 | 5.2 | 4.4 | 66.2 | 47.5 |
| N | W8 | 46 | 1.717 | 0.995 | 0.183 | 0.031 | 4.5 | 3.7 | 60.6 | 45.0 |
| P | BL | 46 | 0.952 | 0.438 | 0.253 | 0.062 | 12.5 | 5.6 | 113.0 | 42.9 |
| P | W2 | 46 | 1.024 | 0.407 | 0.256 | 0.062 | 12.0 | 6.0 | 103.6 | 41.8 |
| P | W4 | 46 | 1.002 | 0.396 | 0.239 | 0.044 | 10.9 | 5.4 | 104.3 | 43.3 |
| P | W8 | 46 | 1.149 | 0.600 | 0.227 | 0.048 | 9.8 | 6.0 | 99.8 | 47.7 |

Changes from baseline were calculated by subtracting each subject's BL values from the appropriate subsequent values. Table 7 summarizes the mean changes from baseline. The mean changes were tested for significance using the one sample t-test against a value of zero. The p value associated with the t statistic is tabulated with the appropriate means, standard deviations and t-values. Statistically significant (p<0.05) results are in bold type with yellow shaded background. Directionally significant (p<0.10) results are in bold type with green shaded background.

In the Normal Sampling direction, there were 2 statistically significant parameter

changes compared to baseline at W8 only. All significant changes were consistent with fewer fine lines by the W8 time point. See the left hand side of Table 2.

In the Parallel Sampling direction, significant changes were seen for 4 parameters at W2, 6 parameters at W4 and all 8 measured parameters at W8. All significant changes were in the direction of smoother, fewer crow's foot wrinkles. See the right hand side of Table 7.

The significantly changed texture parameters suggest about 15% fewer fine lines by the end of the treatment phase. Crow's foot wrinkles became steadily smoother as treatment progressed; at the end of the treatment, the texture parameters indicated smoothing by about 30%.

The 8 wrinkle texture parameters reported in our analysis measure various aspects of the image produced by the replica surface. Generally if there is a substantial smoothing effect, there will be consistent significant changes in several parameters.

Rz and Ra, optical counterparts of classic “stylus” roughness texture parameters:

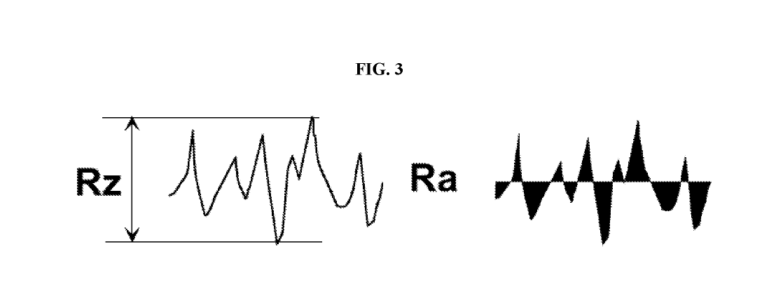

increase with increasing roughness. The diagrams below illustrate the definitions. The profile in the diagram is the brightness profile generated by the angled lighting of the wrinkles on the replica. Note that the amplitude of the profile is not proportional to the depth of the wrinkle but represents the intensity of the shadows behind the wrinkles and highlights in front of the wrinkles. Rz—the maximum difference in luminance value (measured at five equal length segments traversing the sample). Ra—the average deviation of the luminance curve about the mean luminance. (See FIG. 3)

IDL is the length of the line in the Rz diagram above compared to a straight line distance across the surface, increases with roughness of the surface.

FNum is number markers indicative of fine and coarse lines per mm. As lines and creases disappear, FNum decreases.

Spacing is the mean distance in millimeters between adjacent strong shadow features. Sometimes decreases with conversion of deep wrinkles to fine wrinkles (moisturization). Increases with disappearance of wrinkles.

Breadth is proportional to the depth of the wrinkle producing the shadow. May or may not change. Decreases as wrinkles become shallow. This parameter is not sensitive to the number or length of wrinkles.

Shadows parameter is the relative area of shadows cast by all the wrinkles and fine lines in the replica. It is sensitive to both the length and depth of the wrinkles. Decreases with smoothing of the skin.

NumWr is the total number of shadowy features available to calculate spacing and breadth. Generally decreases with smoothing of the skin (fewer visible features).

Questionnaire

Subjects of the clinical study were administered the questionnaire shown in FIG. 1A-FIG. 1C. In summary, after an 8-week clinical study to determine the efficacy of Rapid Repair Healing Cream for skin surface hydration, skin barrier function and improvement of skin texture followed by a consumer self-assessment:

98% agreed that the product was long-lasting

98% reported that their skin was soft

96% agreed that the product was gentle enough for use on face and body

96% reported that their skin was smooth

96% reported that the product felt comfortable on their skin

96% agreed that the product left their skin moisturized

94% agreed that the product was fast-absorbing

94% said their skin felt nourished

92% of the subjects with sensitive skin agreed that the product helped soothe their skin

83% noticed an improvement in their skin tone and texture

Questionnaire—Week 2

Results of the questionnaire shown in FIG. 1A-FIG. 1C after 2 weeks are as follows:

1. The use of this product helped improve my skin tone and texture.

| | | |
|---|---|---|
| I do not feel my skin tone and texture improved | 11 | 23.40% |
| After the first use | 5 | 10.64% |
| Within 24 Hours | 12 | 25.53% |
| After 1 Week | 15 | 31.91% |
| After 2 Weeks | 4 | 8.51 |
| Total Responses | 47 | 100% |

2. I liked the smell of this product.

| | | |
|---|---|---|
| Agree | 26 | 55.32% |
| Disagree | 9 | 19.15% |
| Neutral | 12 | 25.53% |
| Total Responses | 47 | 100% |

Individual Comments are shown in Table 8. Below:

TABLE 8

| Subject Number | 2b. Please explain |
|---|---|
| 2 | the smell is ok, i just don't love it |
| 7 | I don't love the scent but I don't hate it either. It smells like |
| | medicine. |
| 12 | its a little strong and sharp for my personal taste. |
| 18 | smell is ok |
| 19 | a little strong for me |
| 21 | not crazy about smell, but got used to it |
| 28 | When first applied it smells smells ‘woodsy’and masculine |
| | to me . . . but that discipates within a few minutes. If I |
| | know that the product is doing a great job, I can live with |
| | almost any scent |
| 29 | smell was okay |
| 37 | It is a bit strong but wears off okay |
| 40 | Smells like face cream |
| 42 | not a great smell, but not too bad |
| 54 | smell is not strong enough |

3. Did you find the product to be long-lasting?

| | | |
|---|---|---|
| Yes | 43 | 91.49% |
| No | 1 | 2.13% |
| Neutral | 3 | 6.38% |
| Total Responses | 47 | 100% |

Individual responses are shown in Table 9.

TABLE 9

| Subject Number | 3b. Please explain |
|---|---|
| 11 | It feels the same as using my regular everyday product. |
| 46 | My skin didn't feel that different. |
| 49 | since I don't notice any change I really can't respond |
| | to this question |

4. Do you have sensitive skin?

| | | |
|---|---|---|
| Yes | 14 | 29.79% |
| No | 33 | 70.21% |
| Total Responses | 47 | 100% |

4a. If yes, did the use of this product help calm/soothe skin?

| | | |
|---|---|---|
| (Not Answered) | 33 | 70.21% |
| Yes | 12 | 25.53% |
| No | 1 | 2.13% |
| Neutral | 1 | 2.13% |
| Total Responses | 47 | 100% |

Individual responses are shown in Table 10.

TABLE 10

| Subject Number | 4b. Please explain |
|---|---|
| 23 | i didnt notice any calming but it felt smooth and nice |

5. This product is fast absorbing.

| | | |
|---|---|---|
| Agree | 43 | 91.49% |
| Neutral | 4 | 8.51% |
| Total Responses | 47 | 100% |

Individual responses are shown in Table 11.

TABLE 11

| Subject | |
|---|---|
| Number | 5b. Please explain |
| 9 | have to wait awhile before applying makeup |
| 10 | it stay on my skin for a long time |
| 17 | takes some time to fully absorb |
| 22 | It seems to go on my skin easily, but my face does |
| | seem tacky for quite some time afterwards. |

6. This product feels comfortable on my skin.

| | | |
|---|---|---|
| Agree | 43 | 91.49% |
| Disagree | 3 | 6.38% |
| Neutral | 1 | 2.13% |
| Total Responses | 47 | 100% |

Individual responses are shown in Table 12.

TABLE 12

| Subject Number | 6b. Please explain |
|---|---|
| 19 | when I first put it on it feels a little |
| | heave but then feels comfortable |

7. I feel this product is gentle enough for use on face and body.

| | | |
|---|---|---|
| Agree | 44 | 93.62% |
| Disagree | 3 | 6.38% |
| Total Responses | 47 | 100% |

8. This product helped to improve my skin tone and texture.

| | | |
|---|---|---|
| Agree | 33 | 70.21% |
| Disagree | 7 | 14.89% |
| Neutral | 7 | 14.89% |
| Total Responses | 47 | 100% |

Individual responses are shown in Table 13.

TABLE 13

| Subject | |
|---|---|
| Number | 8b. Please explain |
| 7 | I don't feel like it improved my skin tone that |
| | much yet but I think it will alter using it a little |
| | longer. |
| 9 | don't really see any difference |
| 22 | I believe my skin tone is slightly improving, |
| | but the texture is still the same. |
| 31 | didnt notice a change |
| 40 | Looks the same |
| 46 | It neither improved nor damaged my skin as |
| | far as I can tell |
| 52 | it made my skin softer, but did bot rid of ;ines |
| | and wrinkles |

9. My skin felt smooth after use of this product.

| | | |
|---|---|---|
| Agree | 41 | 87.23% |
| Disagree | 3 | 6.38% |
| Neutral | 3 | 6.38% |
| Total Responses | 47 | 100% |

Individual Responses are shown in Table 14.

TABLE 14

| Subject Number | 9b. Please explain |
|---|---|
| 11 | Feels the same as it did before using it. |
| 17 | felt no real difference |
| 46 | It didn't feel smooth, but it didn't feel rough either. |

10. My skin felt soft after use of this product

| | | |
|---|---|---|
| Agree | 41 | 87.23% |
| Disagree | 4 | 8.51% |
| Neutral | 2 | 4.26% |
| Total Responses | 47 | 100% |

Individual Responses are shown in Table 15.

TABLE 15

| Subject Number | 10b. Please explain |
|---|---|
| 11 | Feels the same as it did before using it. |
| 17 | let no real difference |

11. This product left skin moisturized.

| | | |
|---|---|---|
| Agree | 43 | 91.49% |
| Disagree | 2 | 4.26% |
| Neutral | 2 | 4.26% |
| Total Responses | 47 | 100% |

Individual responses are shown in Table 16.

TABLE 16

| Subject Number | 11b. Please explain |
|---|---|
| 11 | [symbol] See answers 9b + 10b |
| 42 | i think it feft the same |

[symbol]

indicates data missing or illegible when filed

12. This product does not leave a sticky or greasy feel on skin.

| | | |
|---|---|---|
| Agree | 39 | 82.98% |
| Disagree | 8 | 17.02% |
| Total Responses | 47 | 100% |

13. This product helped to even skin tone.

| | | |
|---|---|---|
| Agree | 28 | 59.57% |
| Disagree | 12 | 25.53% |
| Neutral | 7 | 14.89% |
| Total Responses | 47 | 100% |

Individual responses are shown in Table 17.

TABLE 17

| Subject | |
|---|---|
| Number | 13b. Please explain |
| 7 | I don't feel like it helped my skin tone that much yet |
| | but feel it will if I keep using the product. |
| 8 | I don't think it helped my skin tone, but it helped the |
| | smoothness, softness and feeling of beng very |
| | moisturized. |
| 21 | worked well moisturizing, cic not notice change on tone |
| 22 | believe that my skin tone is slightly improving, as I see |
| | less blothchy marks. |
| 40 | Skin looks the same |
| 46 | No difference |
| 52 | still have age spots |

14. This product left skin feeling nourished after use of this product.

| | | |
|---|---|---|
| Agree | 43 | 91.49% |
| Disagree | 3 | 6.38% |
| Neutral | 1 | 2.13% |
| Total Responses | 47 | 100% |

Individual responses are shown in Table 18.

TABLE 18

| Subject Number | 14b. Please explain |
|---|---|
| 18 | ok |

15. My skin looked and felt healthy after use of this product.

| | | |
|---|---|---|
| Agree | 39 | 82.98% |
| Disagree | 3 | 6.38% |
| Neutral | 5 | 10.64% |
| Total Responses | 47 | 100% |

Individual responses are shown in Table 19.

TABLE 19

| Subject Number | 15b. Please explain |
|---|---|
| 9 | other then feeling moisturized saw no difference |
| 10 | neither healthy |
| 11 | Since using it for such a short time I can't really say. |
| 17 | made no real difference |
| 42 | same |

16. This product helped to lighten dark/age spots and hyperpigmentation.

| | | |
|---|---|---|
| Agree | 19 | 40.43% |
| Disagree | 13 | 27.66% |
| Not Applicable (I do not have dark/age | 8 | 17.02% |
| spots or hyperpigmentation) | | |
| Neutral | 7 | 14.89% |
| Total Responses | 47 | 100% |

Individual responses are shown below in Table 20.

TABLE 20

| Subject Number | 16b. Please explain |
|---|---|
| 2 | i see a very slight difference |
| 7 | I don't feel like it has helped lighten my dark/age spots |
| | yet but I feel like it will after using the product longer. |
| 18 | ok |
| 19 | I dont feel that the age spots have lightened |
| 21 | did not notice |
| 22 | Initially I saw a lot of spots and blothchy areas, but it |
| | has been improving for the past two weeks. |
| 50 | I still have some dark spots |

17. How likely are you to purchase this product?

| | | |
|---|---|---|
| Somewhat Likely | 15 | 31.91% |
| Likely | 23 | 48.94% |
| Neutral | 4 | 8.51% |
| Not at All | 5 | 10.64% |
| Total Responses | 47 | 100% |

Individual responses are shown below in Table 21.

TABLE 21

| Subject | |
|---|---|
| Number | 17b. Please explain |
| 9 | not sure didn't do much then other products |
| 11 | Again, it hasn't been enough time to tell |
| 36 | maybe |
| 52 | worked well as moisturizer bit did not |
| | improve age spots |

18. As we truly value your opinion, we welcome any additional comments/suggestions that should be considered to further enhance product benefits and/or usage. Please use the space below for any additional comments.

Individual responses are shown below in Table 22.

TABLE 22

| Subject | |
|---|---|
| Number | Response |
| 1 | my skin feels softer and smoother |
| 2 | i feel the product is a nice product |
| 3 | na |
| 4 | na |
| 6 | the cream works well |
| 7 | I like the product. The scent can be a little nicer but it |
| | does smell like a healing cream. I feel with continued |
| | use it will make my skin tone and age spots better. |
| 8 | I love this product as a skin moisturizer and would use it |
| | everywhere on my body if I could. My son gave me |
| | a kiss and said ‘Mom, your cheeks are so soft!’ Usually |
| | my skin is very tight and dry on my face in the winter, |
| | but now it's soft and moisturized. |
| 9 | no other comments |
| 10 | i don't like the smell of the produt. |
| 11 | No comment at this time. |
| 12 | I like how it feels when appliying. It feels thick in my |
| | hands, but feels like a thin, protective layer immediately |
| | on skin. |
| 13 | this product was very smoooth and creamy. i love the |
| | scent also. |
| 14 | wouldm like to know the name of this product so that I |
| | look for it on line to see what store in our area sells it so |
| | that I can buy it when the study is over on Feb. 12, 2015. |
| 17 | felt it took time to fully absorb into the skin, saw no real |
| | difference in skin tone or texture |
| 18 | i like it |
| 19 | I would perfer a less floral smell |
| 20 | none |
| 21 | loved for moisturizing sensitive skin, would love to keep |
| | using |
| 22 | I believe that the product would be easier to apply if it |
| | were in a pump container. |
| 23 | I lived the feel of this although I did not yet notice |
| | lightening |
| 25 | i like the product and would use it and recommend it |
| 26 | product absorbed beter on arm |
| 27 | I really like this product and the smell it beautiful |
| 28 | I have noticed that the lines between my eyebrows have |
| | become less apparent and my face seems brighter. |
| 29 | made my skin feel moist |
| 30 | was smooth tp apply |
| 31 | The smell is a major turn off. Otherwise product was |
| | quite pleasent |
| 34 | i love this cream |
| 36 | product was nice and it smelled good. I did not notice a |
| | difference |
| 37 | Too sticky feeling |
| 38 | I like the product but not the smell. It smells like soap |
| 39 | I like the product |
| 40 | I have no suggestions |
| 41 | i like the product |
| 42 | I thought this product was dense |
| 43 | good product |
| 44 | love te feel of product made my skin feel nourished and |
| | soft imediatley |
| 45 | change smell of product |
| 46 | Initially the product stung my face, but after the second |
| | application, my skin must have acclimated as it was no |
| | longer a problem. I didn't like the smell and prefer |
| | products on my face to be unscented. |
| 47 | i like proudut its nice and erry smooth |
| 48 | I like the product but do not like the smell very much. |
| 49 | not crazy about the fragrance |
| 50 | I like this product very much, it doesn't leave a thick film |
| | on my face after putting it on. |
| 51 | the cream makes my skin feel softer and the soap makes |
| | my skin feel more moistrerized |
| 52 | did not ;ike smell, it smelt like Noxema |
| 53 | I would like it better if it were not quite as thick, and if it |
| | were unscented or had a lighter scent. Out of everything, I |
| | dislike the scent the mostl |
| 54 | it should be with little more frangnance |

Questionnaire—Week 4

Results of the questionnaire shown in FIG. 1A-FIG. 1C after 4 weeks are as follows:

1. The use of this product helped improve my skin tone and texture.

| | | |
|---|---|---|
| I do not feel my skin tone and texture improved | 8 | 17.02% |
| After the first use | 5 | 10.64% |
| Within 24 Hours | 5 | 10.64% |
| After 1 Week | 9 | 19.15% |
| After 2 Weeks | 12 | 25.53% |
| After 3 Weeks | 6 | 12.77% |
| After 4 Weeks | 2 | 4.26% |
| Total Responses | 47 | 100% |

2. I liked the smell of this product.

| | | |
|---|---|---|
| Agree | 30 | 63.83% |
| Disagree | 13 | 27.66% |
| Neutral | 4 | 8.51% |
| Total Responses | 47 | 100% |

Individual Comments are shown in Table 23. Below:

TABLE 23

| Subject | |
|---|---|
| Number | 2b. Please explain |
| 2 | i dont think that this product has a strong smell, |
| | so really nothing to ike |
| 7 | Smells like medicine |
| 9 | not overly scented |
| 28 | smelled ‘woodsy’/natural. would rather have this |
| | scent then perfumy |

3. Did you find the product to be long-lasting?

| | | |
|---|---|---|
| Yes | 44 | 93.62% |
| No | 2 | 4.26% |
| Neutral | 1 | 2.13% |
| Total Responses | 47 | 100% |

Individual responses are shown in Table 24.

TABLE 24

| Subject | |
|---|---|
| Number | 3b. Please explain |
| 52 | it improved skin texture but was not long lasting |

4. Do you have sensitive skin?

| | | |
|---|---|---|
| Yes | 14 | 29.79% |
| No | 33 | 70.21% |
| Total Responses | 47 | 100% |

4a. If yes, did the use of this product help calm/soothe skin?

| | | |
|---|---|---|
| (Not Answered) | 33 | 70.21% |
| Yes | 12 | 25.53% |
| Neutral | 2 | 4.26% |
| Total Responses | 47 | 100% |

Individual responses are shown in Table 25.

TABLE 25

| Subject Number | 4b. Please explain |
|---|---|
| 23 | skin feels moisturized but calmed? not sure |
| 53 | It didn't calm it but didn't bother it. |

5. This product is fast absorbing.

| | | |
|---|---|---|
| Agree | 43 | 91.49% |
| Neutral | 4 | 8.51% |
| Total Responses | 47 | 100% |

6. This product feels comfortable on my skin.

| | | |
|---|---|---|
| Agree | 45 | 95.74% |
| Neutral | 2 | 4.26% |
| Total Responses | 47 | 100% |

Individual responses are shown in Table 26.

TABLE 26

| Subject Number | 6b. Please explain |
|---|---|
| 10 | I feel it is a lite too heavy for my skin. |
| 17 | feels greasy does not absorb fully |

7. I feel this product is gentle enough for use on face and body.

| | | |
|---|---|---|
| Agree | 46 | 97.87% |
| Disagree | 1 | 2.13% |
| Total Responses | 47 | 100% |

8. This product helped to improve my skin tone and texture.

| | | |
|---|---|---|
| Agree | 35 | 74.47% |
| Disagree | 7 | 14.89% |
| Neutral | 5 | 10.64% |
| Total Responses | 47 | 100% |

Individual responses are shown in Table 27.

TABLE 27

| Subject Number | 8b. Please explain |
|---|---|
| 9 | skin felt hydrated but didn't notice any |
| | evenness or even tone |
| 10 | I haven't see any changes |
| 21 | i didnt see a difference with tone |
| 49 | don't notice much difference |
| 52 | not long lasating |

9. My skin felt smooth after use of this product.

| | | |
|---|---|---|
| Agree | 45 | 95.74% |
| Disagree | 2 | 4.26% |
| Total Responses | 47 | 100% |

10. My skin felt soft after use of this product

| | | |
|---|---|---|
| Agree | 44 | 93.62% |
| Disagree | 2 | 4.26% |
| Neutral | 1 | 2.13% |
| Total Responses | 47 | 100% |

Individual Responses are shown in Table 28.

TABLE 28

| Subject Number | 10b. Pease explain |
|---|---|
| 17 | really felt no difference |

11. This product left skin moisturized.

| | | |
|---|---|---|
| Agree | 42 | 89.36% |
| Disagree | 4 | 8.51% |
| Neutral | 1 | 2.13% |
| Total Responses | 47 | 100% |

Individual responses are shown in Table 29.

TABLE 29

| Subject Number | 11b. Please explain |
|---|---|
| 28 | after about 5 hrs of wear, my face became oily |

12. This product does not leave a sticky or greasy feel on skin.

| | | |
|---|---|---|
| Agree | 38 | 80.85% |
| Disagree | 6 | 12.77% |
| Neutral | 3 | 6.38% |
| Total Responses | 47 | 100% |

Individual responses are shown in Table 30.

TABLE 30

| Subject Number | 12b. Please explain |
|---|---|
| 18 | ok |
| 19 | when first appled it felt greasy but after a few |
| | min. it is fine |
| 22 | It took a little longer than my ususal product |
| | for the test product to absorb. |

13. This product helped to even skin tone.

| | | |
|---|---|---|
| Agree | 28 | 59.57% |
| Disagree | 16 | 34.03% |
| Neutral | 3 | 6.38% |
| Total Responses | 47 | 100% |

Individual responses are shown in Table 31.

TABLE 31

| Subject Number | 13b. Please explain |
|---|---|
| 2 | I think there is only a slight difference in my skin tone. |
| 19 | no change |
| 22 | I am not noticing any difference in my skin tone. |

14. This product left skin feeling nourished after use of this product.

| | | |
|---|---|---|
| Agree | 41 | 85.11% |
| Disagree | 4 | 8.51% |
| Neutral | 2 | 4.26% |
| Total Responses | 47 | 100% |

Individual responses are shown in Table 32.

TABLE 32

| Subject Number | 14b. Please explain |
|---|---|
| 17 | no difference |
| 28 | as stated previously, after about 5 hrs, my face was oily |

15. My skin looked and felt healthy after use of this product.

| | | |
|---|---|---|
| Agree | 40 | 85.11% |
| Disagree | 3 | 6.38% |
| Neutral | 4 | 8.51% |
| Total Responses | 47 | 100% |

Individual responses are shown in Table 33.

TABLE 33

| Subject Number | 15b. Please explain |
|---|---|
| 9 | felt skin was hydrated did not even tone |
| 17 | no real difference |
| 42 | looked and felt the same |
| 46 | I didn't notice a change. My skin was niether |
| | healthier or worse off after use. |

16. This product helped to lighten dark/age spots and hyperpigmentation.

| | | |
|---|---|---|
| Agree | 18 | 38.30% |
| Disagree | 16 | 34.04% |
| Not Applicable (I do not have dark/age | 6 | 12.77% |
| spots or hyperpigmentation) | | |
| Neutral | 7 | 14.89% |
| Total Responses | 47 | 100% |

Individual responses are shown below in Table 34.

TABLE 34

| Subject Number | 16b. Please explain |
|---|---|
| 2 | I really do not see much of a difference |
| 3 | didnt like the sent |
| 8 | I don't see a BIG improvement in lightening of my skin |
| | (subtle improvement) but I would use this consistently |
| | to achieve that effect. |
| 13 | not as of yet |
| 22 | The dark spots on my face have not changed since I |
| | started using this product. |
| 23 | have not noticed a lightening |
| 27 | not sure |

17. How likely are you to purchase this product?

| | | |
|---|---|---|
| Somewhat Likely | 11 | 23.40% |
| Likely | 25 | 53.19% |
| Neutral | 7 | 14.89% |
| Not at All | 4 | 8.51% |
| Total Responses | 47 | 100% |

Individual responses are shown below in Table 35.

TABLE 35

| Subject Number | 17b. Please explain |
|---|---|
| 9 | only purchase for hydratin purposes only |
| 11 | still not seeing any changes |
| 17 | has not been better than others |
| 28 | I don't like that my face is getting oily in the afternoon |
| 42 | not at this point |
| 48 | don't like the smell |
| 53 | The smell is AWFUL. I might purchase it if it had a light |
| | or no scent, but unless it worked miracles on my skin, I |
| | would most likely not buy it because of the scent. |

18. As we truly value your opinion, we welcome any additional comments/suggestions that should be considered to further enhance product benefits and/or usage. Please use the space below for any additional comments.

Individual responses are shown below in Table 36.

TABLE 36

| Subject Number | Response |
|---|---|
| 1 | this is an inovative product. I like the results |
| 2 | I like the product as far as a moisturizer and do think |
| | that the line looks better. |
| 3 | did not like thesent |
| 4 | Nothing |
| 6 | this product really makes the skin feel soft, nice smell, |
| | hydrates skin |
| 7 | The scent can be improved but overall I like the product. |
| 8 | I love, love love this product. I would purchase it right |
| | away if it was available. I would tell all my friends about |
| | it. My skin has never felt softer or more moisturized. |
| | The product absorbs quickly, smells good, and my skin |
| | is finally not tight. Love! |
| 9 | felt great for hydration both for face and body did not |
| | help brown or dark spots at all |
| 10 | at first the smell bothers me, now I'm getting use to |
| | it . . . . |
| 11 | i like the feel of product and waiting to see if there are |
| | any results. |
| 12 | My skin feels really smooth after using this product. I |
| | would most definitely buy it! |
| 13 | i like the product very much .it goes on smooth and stays |
| | on for along time. |
| 14 | When I find out the name of this product, I will go out & |
| | buy it right away & throw out the products I am using. |
| 17 | the smell is not that pleasant and does not seem to |
| | absorb fully on my skin . . . may be good for those who |
| | have very dry skin. I do not the feel of greasy skin which |
| | this gives me |
| 18 | ok |
| 19 | the smell is to stonge for my taste . . . thanks! |
| 20 | none |
| 21 | no comment |
| 22 | After using this product for a month I feel a pump bottle |
| | would be easier and quicker to use. |
| 23 | I have found that using more and massaging it in makes |
| | a bigger difference |
| 25 | i would use this product all the time |
| 26 | product nakes my skin soft and feels younger i would |
| | deff buy product very gentle to my sensitve skin |
| 27 | I really like this product . . . no greasy . . . great smell |
| 28 | the first 2 weeks my face was not oily, but now it is |
| | becoming greasy in the afternoon, around 2 or |
| | 3 . . . YUCK |
| 29 | made my skin soft |
| 30 | leaves my skin smooth |
| 31 | The smell was a major turn off |
| 34 | i love this cream |
| 36 | no comment except that it does go on smooth |
| 37 | Do not like the sticky feeling of this product |
| 38 | I Like the product it makes my face feel smooth and |
| | moisturized but needs improvment in the smell |
| 39 | no |
| 40 | i enjoy the way the product smells, so i woundn't change |
| | the intensity or the cool affect. My skin diffenetly has |
| | improved with out any irritation. I would love to use this |
| | product on a daily use. |
| 41 | like the product |
| 42 | product was a little dense |
| 43 | leavee skin soft |
| 44 | I love this product. I would use it all over mybody it |
| | smells great and nourished my skin |
| 45 | chamgethe scent |
| 46 | My skin stopped stinging after the initial use, but I do |
| | not like to use fragrance on my face. I like how the |
| | product applies, meaning it's not greasy or oily, but my |
| | skin does not feel particularly moisturized after use. |
| 47 | my skin fills soft after use the procud |
| 48 | i like the product but not the smell |
| 49 | skin feels softer definitely; don't notice change in dark |
| | spots. Not crazy about fragrence |
| 50 | I like this product very much. Leaves my skin soft and |
| | not sticky. |
| 51 | it leaves my skin feeling very smooth |
| 52 | did not like scent of this product, it smelled like |
| | Noxzema! |
| 53 | It could be a little thinner, and the scent really needs to |
| | be changed! |
| 54 | I like the thickness of the cream |

Questionnaire—Week 8

Results of the questionnaire shown in FIG. 1A-FIG. 1C after 8 weeks are as follows:

1. The use of this product helped improve my skin tone and texture.

| | | |
|---|---|---|
| I do not feel my skin tone and texture improved | 9 | 19.15% |
| After the first use | 4 | 8.51% |
| Within 24 Hours | 6 | 12.77% |
| After 1 Week | 12 | 25.53% |
| After 2 Weeks | 6 | 12.77% |
| After 3 Weeks | 4 | 8.51% |
| After 4 Weeks | 2 | 4.26% |
| After 6 Weeks | 2 | 4.26% |
| After 8 Weeks | 2 | 4.26% |
| Total Responses | 47 | 100% |

2. I liked the smell of this product.

| | | |
|---|---|---|
| Agree | 33 | 70.21% |
| Disagree | 9 | 19.15% |
| Neutral | 5 | 10.61% |
| Total Responses | 47 | 100% |

Individual Comments are shown in Table 37. Below:

TABLE 37

| Subject Number | 2b. Please explain |
|---|---|
| 2 | not much of a smel |
| 6 | scent should be improved |
| 7 | A little medicine smelling but not horrible. |
| 17 | was not bad or good |
| 21 | did not care much for the smell |

3. Did you find the product to be long-lasting?

| | | |
|---|---|---|
| Yes | 46 | 97.87% |
| No | 1 | 2.13% |
| Total Responses | 47 | 100% |

4. Do you have sensitive skin?

| | | |
|---|---|---|
| Yes | 12 | 25.53% |
| No | 35 | 74.47% |
| Total Responses | 47 | 100% |

4a. If yes, did the use of this product help calm/soothe skin?

| | | |
|---|---|---|
| (Not Answered) | 35 | 74.47% |
| Yes | 11 | 23.40% |
| Neutral | 1 | 2.13% |
| Total Responses | 47 | 100% |

Individual responses are shown in Table 38.

TABLE 38

| Subject Number | 4b. Please explain |
|---|---|
| 53 | didn't calm but didn't bother either |

5. This product is fast absorbing.

| | | |
|---|---|---|
| Agree | 44 | 93.62% |
| Disagree | 1 | 2.13% |
| Neutral | 2 | 4.26% |
| Total Responses | 47 | 100% |

Individual responses are shown in Table 39.

TABLE 39

| Subject Number | 5b. Please explain |
|---|---|
| 23 | some pilling |
| 53 | it took a little while, it wasn't immediate |

6. This product feels comfortable on my skin.

| | | |
|---|---|---|
| Agree | 45 | 95.74% |
| Neutral | 2 | 4.26% |
| Total Responses | 47 | 100% |

7. I feel this product is gentle enough for use on face and body.

| | | |
|---|---|---|
| Agree | 45 | 95.74% |
| Disagree | 1 | 2.13% |
| Neutral | 1 | 2.13% |
| Total Responses | 47 | 100% |

8. This product helped to improve my skin tone and texture.

| | | |
|---|---|---|
| Agree | 39 | 82.98% |
| Disagree | 4 | 8.51% |
| Neutral | 4 | 8.51% |
| Total Responses | 47 | 100% |

Individual responses are shown in Table 40.

TABLE 40

| Subject Number | 8b. Please explain |
|---|---|
| 9 | if it did very minor |
| 11 | I really couldn't see any noticeable changes |
| 21 | heped with texture by moisturizing, not really tone |
| 52 | did not improve tone or texture |

9. My skin felt smooth after use of this product.

| | | |
|---|---|---|
| Agree | 45 | 95.74% |
| Disagree | 2 | 4.26% |
| Total Responses | 47 | 100% |

Individual responses are shown in Table 41.

TABLE 41

| Subject Number | 9b. Please explain |
|---|---|
| 28 | to me oily is not smooth |
| 48 | could be smoother |

10. My skin felt soft after use of this product

| | | |
|---|---|---|
| Agree | 46 | 97.87% |
| Disagree | 1 | 2.13% |
| Total Responses | 47 | 100% |

Individual Responses are shown in Table 42.

TABLE 42

| Subject Number | 10b. Please explain |
|---|---|
| 48 | could be softer |

11. This product left skin moisturized.

| | | |
|---|---|---|
| Agree | 45 | 95.74% |
| Disagree | 1 | 2.13% |
| Neutral | 1 | 2.13% |
| Total Responses | 47 | 100% |

Individual responses are shown in Table 43.

TABLE 43

| Subject Number | 11b. Please explain |
|---|---|
| 48 | it would have liked a little more moisure |

12. This product does not leave a sticky or greasy feel on skin.

| | | |
|---|---|---|
| Agree | 41 | 87.23% |
| Disagree | 5 | 10.64% |
| Neutral | 1 | 2.13% |
| Total Responses | 47 | 100% |

Individual responses are shown in Table 44.

TABLE 44

| Subject Number | 12b. Please explain |
|---|---|
| 53 | it feels a little greasy at first, |
| | but then it goes away |

13. This product helped to even skin tone.

| | | |
|---|---|---|
| Agree | 33 | 70.21% |
| Disagree | 11 | 23.40 |
| Neutral | 3 | 6.38% |
| Total Responses | 47 | 100% |

Individual responses are shown in Table 45.

TABLE 45

| Subject Number | 13b. Please explain |
|---|---|
| 11 | Again, I could notice and visual changes |
| 28 | i see a very slight change |
| 52 | did not notice any difference |

14. This product left skin feeling nourished after use of this product.

| | | |
|---|---|---|
| Agree | 44 | 93.62% |
| Disagree | 1 | 2.13% |
| Neutral | 2 | 4.26% |
| Total Responses | 47 | 100% |

Individual responses are shown in Table 46.

TABLE 46

| Subject Number | 14b. Please explain |
|---|---|
| 11 | I liked the feel of the product on my |
| | skin I couldn't say it felt nourished |
| 48 | it was nothing special |

15. My skin looked and felt healthy after use of this product.

| | | |
|---|---|---|
| Agree | 39 | 82.98% |
| Disagree | 2 | 4.26% |
| Neutral | 6 | 12.77% |
| Total Responses | 47 | 100% |

Individual responses are shown in Table 47.

TABLE 47

| Subject Number | 15b. Please explain |
|---|---|
| 9 | was moisturized but no change in texture |
| 11 | No noticeable changes |
| 17 | made no real change |
| 48 | no change |
| 49 | no noticable change in appearance, just in feel |
| 52 | skon felt softer but did not improve age spot, |
| | wrinkles |

16. This product helped to lighten dark/age snots and hyperpigmentation.

| | | |
|---|---|---|
| Agree | 21 | 44.68% |
| Disagree | 10 | 21.28% |
| Not Applicable (I do | 11 | 23.40% |
| not have dark/age spots | | |
| or hyperpigmentation) | | |
| Neutral | 5 | 10.64% |
| Total Responses | 47 | 100% |

Individual responses are shown below in Table 48.

TABLE 48

| Subject Number | 16b. Please explain |
|---|---|
| 8 | I did not see a lightening of age spots on my face. I |
| | don't have dark spots but did not see any difference |
| | with them. |
| 13 | really didint chamge that much to my age spots |
| 22 | Since I have some hyperpigmentaton on my face I |
| | look at the areas daily, and I did not notice any |
| | specific changes. |
| 23 | did not notice too much difference. |
| 40 | saw some fading but not much |

17. How likely are you to purchase this product?

| | | |
|---|---|---|
| Somewhat Likely | 11 | 23.40% |
| Likely | 27 | 57.45% |
| Neutral | 4 | 8.51% |
| Not at All | 5 | 10.64% |
| Total Responses | 47 | 100% |

Individual responses are shown below in Table 49.

TABLE 49

| Subject Number | 17b. Please explain |
|---|---|
| 11 | I like the product but to the naked eye I didn't see any |
| | changes |
| 48 | nothing special about it. don't really like the smell. |
| 49 | didn't see that much change |
| 53 | I like how it worked but I HATE the scent! |

18. As we truly value your opinion, we welcome any additional comments/suggestions that should be considered to further enhance product benefits and/or usage. Please use the space below for any additional comments.

Individual responses are shown below in Table 50.

TABLE 50

| Subject | |
|---|---|
| Number | Response |
| 1 | I loved the smell, this product definitely helped moisturize my skin, didnt leave |
| | my skin with the sticky feeling |
| 2 | [symbol] thought this product was good for my skin. |
| 3 | didnt like the smell |
| 4 | [symbol] |
| 6 | [symbol] liked this product but the scent needs to beimproved |
| 7 | I liked the product very much. It could smell a little better but it wasn't |
| | horrible. It actually smelled like a healing cream. My face feels soft, |
| | moisterized and smooth from using it. My face was not dry while using it. |
| 8 | I love this product for it's moisturizing effect on my face. Long lasting and |
| | made my skin very soft. I would purchase this to use on my face and my |
| | whole body [symbol] |
| 9 | the product was good for moisturizing skin but not for texture there are better |
| | products on the market but I could see this used as a body lotion |
| 10 | [symbol] didn'like the smel of the product . . . |
| 11 | This product smells and feels good on my skin. After using it for many weeks I |
| | really couldn't see any noticeable changes |
| 12 | I would be very likely to purchase this product, except they don't tell us the |
| | name of the product here where its tested. I think it is a wondrful product. For |
| | me, especially, I noticed it helped with the wrinkles around my mouth, in |
| | addition to my eye |
| 13 | [symbol] really liked the product alot [symbol] it went on my skin very smoothly. |
| | loved the scent of it [symbol] wanted to keep it. thank you . . . |
| 14 | This product also removes any bruising to the skin & it heats any pain area as |
| | will. I would defina [symbol] y buy this product once I know the name of it & |
| | where it is sold. |
| 17 | felt a little greasy [symbol] did not really change my skin |
| 18 | This program ws very good |
| 19 | I did not like the smell. howeve the product it self was great and I would buy |
| | the product if it had a different smell. Thank you |
| 20 | none |
| 21 | [symbol] ked product |
| 22 | I believe it would be easier to use the product if it were in a pump container. |
| 23 | face really feels smooth and moisturized. |
| 25 | [symbol] t applys easy amd smells good |
| 26 | very gentle soft mosturizing product good i liked |
| 27 | [symbol] liked this product . . . it was thick and the smell was very nice |
| 28 | the first 3 weeks or so I liked what I saw a change in my skin. However, at |
| | about week 4 or so . . . the product clogged my pores and made my face vary |
| | oily. I cut back on the amount I was applying, but my face seem to have a |
| | coated feeling. |
| 29 | lotion made skin soft |
| 30 | made my skin smooth |
| 31 | The smell is a major turn off |
| 34 | [symbol] love this product it made my skin feel very soft |
| 36 | na |
| 37 | consistency a little too thick |
| 38 | after 1 mo [symbol] th of use [symbol] got used to the smell of thencream and |
| | afterwards [symbol] t didnt smell bad any longer |
| 39 | [symbol] like the product |
| 40 | [symbol] don [symbol] t have additional comment |
| 41 | good product |
| 42 | THis product took about 6 weeks to show any noticeable change on my skin. |
| | BUt all in all, it was a good product. |
| 43 | very good product hope to see it on the market |
| 44 | [symbol] loved thosproduct it was gentle and smelled great |
| 45 | nice product |
| 46 | I was not impressed at first use of the product, but over time my skin felt |
| | much softer and moisturized. I grew to like the product and am sorry not to |
| | have anymore. |
| 47 | the pru [symbol] duct fills nice one my face abd is smooth |
| 48 | the product was pretty good and I think slot of people would like it. It just |
| | isn't right for me. |
| 49 | not crazy about fragrence |
| 50 | This product was very good. Kept my skin soft and smooth. After applying to |
| | my skin it dried so fast. I love that in a cream |
| 51 | The product left my skin feeling very smooth and moisterised |
| 52 | product scent smelled like Noxzema. Softened skin. bur that was it [symbol] |
| 53 | It is very thick and smells pretty awful, but seemed to work well on wrinkles. |
| | Maybe if it were not quite as thick it would absorb more quickly. And the |
| | scent really is horrible! |
| 54 | I wish it could diminish frown lines and crow feet |

[symbol]

indicates data missing or illegible when filed

A statistical analysis of the questionnaire responses is shown below in Table 51.

TABLE 51

| | Week 2 | | Week 4 | | Week 8 | |
|---|---|---|---|---|---|---|
| | Z- | | Z- | | Z- | |
| Question (n = 47) | Score | Significant | Score | Significant | Score | Significant |
| 2. I liked the smell of the product. | 2.48 | Yes | 2.48 | Yes | 3.50 | Yes |
| 3. Did you find the product to be long-lasting? | 6.13 | Yes | 6.13 | Yes | 6.56 | Yes |
| 4. Do you have sensitive skin? 4a. If yes, did | 2.94 | Yes | 3.21 | Yes | 3.18 | Yes |
| the use of this product help calm/soothe skin? | | | | | | |
| 5. This product is fast absorbing. | 6.27 | Yes | 5.69 | Yes | 6.27 | Yes |
| 6. This product feels comfortable on my skin. | 5.83 | Yes | 6.56 | Yes | 6.27 | Yes |
| 7. I feel this product is gentle enongh for use | 5.98 | Yes | 6.56 | Yes | 6.42 | Yes |
| on face and body. | | | | | | |
| 8. This product helped to improve skin tone | 3.79 | Yes | 4.08 | Yes | 5.11 | Yes |
| and texture. | | | | | | |
| 9. My skin felt smooth after use of this | 5.54 | Yes | 6.27 | Yes | 6.56 | Yes |
| product. | | | | | | |
| 10. My skin felt soft after use of this product. | 5.40 | Yes | 6.13 | Yes | 6.71 | Yes |
| 11. This product left skin moisturized. | 5.98 | Yes | 5.54 | Yes | 6.42 | Yes |
| 12. This product does not leave a sticky or | 4.52 | Yes | 4.67 | Yes | 5.25 | Yes |
| greasy feel on skin. | | | | | | |
| 13. This product helped to even skin tone. | 2.33 | Yes | 1.75 | No | 3.21 | Yes |
| 14. This product left skin feeling nourished | 5.83 | Yes | 5.40 | Yes | 6.27 | Yes |
| after use of this product. | | | | | | |
| 15. My skin looked and felt healthy after use | 5.25 | Yes | 5.40 | Yes | 5.40 | Yes |
| of this product. | | | | | | |
| 16. This product helped to lighten dark/age | 0.96 | No | 0.31 | No | 1.83 | No |
| spots and hyperpigmentation. | | | | | | |
| 17. How likely are you to purchase this | 4.81 | Yes | 4.67 | Yes | 4.81 | Yes |
| product? | | | | | | |

*Z-test was not performed for Q1 and Q4 as the responses are not applicable for z-test

Daily Diaries

There were no comments recorded on the Daily Diary that were related to reactions or symptoms perceived during test material use.

Adverse Events

There was one, irrelevant adverse event reported during the study as shown in Table 52.

TABLE 52

| | | Description of Event | | | | Relationship |
|---|---|---|---|---|---|---|
| Subject | Study | | Observations | | Action | to Test |
| # | Day | Subject Reported: | at laboratory: | Severity | Taken | Material |
| 32 | 11 | Slipped on icy stairs | Not | Severe | Discontinued | Definitely |
| | | and broke left leg | Applicable | | | Not |

### Clinical Study Conclusions

Under the conditions of this study and in this population, the test material identified as June Jacobs Healing Cream TLB1-113/8 demonstrated a potential to increase skin hydration and improve skin barrier function and skin texture during an 8-week use period.

Statistically significant improvements in Corneometer® measurements of skin surface hydration were observed 24 hours following a single application. Increases in Corneometer® measurements were statistically greater at sites treated with the test material compared to untreated sites.

Statistically significant decreases in DermaLab® measurements of transepidermal water loss were observed 24 hours following a single application and following 2, 4, and 8 weeks of daily treatment, indicating improved skin barrier function. Decreases in TEWL measurements were statistically greater at sites treated with the test material compared to untreated sites at all evaluation time points.

Analysis of Silflo Skin Replicas was performed by David L. Miller, Ph.D. at BioNET, Incorporated. As noted in the attached report, “The significantly changed texture parameters suggest about 15% fewer fine lines by the end of the treatment phase. Crow's foot wrinkles became steadily smoother as treatment progressed; at the end of the treatment, the texture parameters indicated smoothing by about 30%.”

Questionnaires completed by subjects following 2, 4, and 8 weeks of use indicated that a statistically significant portion of the test population agreed with the following at the time point indicated with an “X” in table 53 below.

TABLE 53

| Question | Week 2 | Week 4 | Week 8 |
|---|---|---|---|
| liked the smell of this product | X | X | X |
| found the product to be long-lasting | X | X | X |
| the product help calm/soothe skin | X | X | X |
| (sensitive skin subjects only) | | | |
| product is fast absorbing | X | X | X |
| product feels comfortable on skin | X | X | X |
| product is gentle enough for use | X | X | X |
| on face and body | | | |
| product helped to improve skin | X | X | X |
| tone and texture | | | |
| skin felt smooth after use of this | X | X | X |
| product | | | |
| skin felt soft after use if this | X | X | X |
| product | | | |
| product left skin moisturized | X | X | X |
| product does not leave a sticky or | X | X | X |
| greasy feel on skin | | | |
| product helped to even skin tone | X | | X |
| product left skin feeling nourished | X | X | X |
| after use | | | |
| skin looked and felt healthy after | X | X | X |
| use of this product | | | |
| likely to purchase this product | X | X | X |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A composition for treating or preventing erythema and/or radiation dermatitis, the composition comprising at least two skin soothing agents, a blend of at least two antioxidants, at least one skin conditioning agent, and a cosmetically acceptable vehicle.

2. The composition of claim 1, wherein the soothing agents make up about 2% to 15% or 5% to 10% of the composition by weight.

3. The composition of claim 1, wherein the at least two skin soothing agents are selected from the group consisting of aloe vera, phytostearyl canola glycerides, allantoin, caprylic/capric triglyceride, bisabolol, colloidal oatmeal, Peucedanum ostruthium leaf extract, a long chain alkyl benzoate such as C₁₂₋₁₅ alkyl benzoate, C₁₆₋₁₇ alkyl benzoate, stearyl benzoate, isostearyl benzoate, ethylhexyl benzoate, octyldodecyl benzoate, and a ceramide.

4. The composition of claim 3, wherein the at least two skin soothing agents comprise about 1.5% of the composition by weight.

5. The composition of claim 3, wherein the composition comprises at least four skin soothing agents.

6. The composition of claim 3, wherein the at least four skin soothing agents comprise about 1.5% to 2% of the composition by weight.

7. The composition of claim 1, wherein the skin soothing agents comprise at least two or at least four of the following: phytostearyl canola glycerides, bisabolol, Peucedanum ostruthium leaf extract, and ceramide AP.

8. The composition of claim 7, wherein the composition comprises a blend of at least four antioxidants selected from Leontopodium alpinum extract, Aspalathus linearis (red tea) leaf extract, Camellia sinensis (green and white Tea) leaf extract, Lycium varbarum (Goji Berry) fruit extract, Punica granatum (Pomegranate) extract, and Vitis vinifera (Grape) seed extract.

9. The composition of claim 8, wherein the blend comprises Leontopodium alpinum extract.

10. The composition of claim 8, wherein the blend comprises about 0.8% to 1.2% of the composition by weight.

11. The composition of claim 1, wherein the at least one skin conditioning agent is selected from Butyrospermum parkii (Shea) butter, triolein, and argania spinosa kernel oil.

12. The composition of claim 11, wherein the at least one skin conditioning agent is selected from triolein, and argania spinosa kernel oil.

13. The composition of claim 12, wherein the at least one skin conditioning agent comprises from about 2% to 10% or from about 2% to 8% or from about 8% to 10% of the composition by weight.

14. The composition of claim 1, further comprising at least one emulsifier.

15. The composition of claim 14, wherein the at least one emulsifier is selected from oleic acid, palmitic acid, glyceryl stearate, cetearyl alcohol, xanthan gum, linoleic acid, and lecithin.

16. The composition of claim 1, wherein the composition further comprises one or more of a skin moisturizing agent, a fragrance, an anti-microbial agent, a sunscreen, a pH modulator, and a preservative.

17. The composition of claim 1, wherein the cosmetically acceptable vehicle is an aqueous vehicle.

18. The composition of claim 17, wherein the aqueous vehicle is water.

19. A method for treating or preventing erythema and/or radiation dermatitis in a subject in need thereof, the method comprising topically applying the composition of claim 1 to an affected area of the subject's skin in an amount sufficient to cover the affected area with the composition.

20. The method of claim 19, wherein the subject in need thereof is a human subject.

21. The method of claim 20, wherein the human subject has received radiation therapy.

22. The method of claim 21, wherein the human subject is a breast cancer patient.

23. The method of claim 22, further comprising reapplying the composition to the affected area once or twice daily for a period of time ranging from one to six weeks or from two to twelve weeks.

24. The method of claim 23, wherein the affected area is an area that was exposed to radiation.
